(19) United States
(12) Patent Application Publication
(10) Pub. No.: US 2019/0189277 A1
(43) Pub. Date: Jun. 20, 2019

(54) MEDICAL SUPPORT SYSTEM, INFORMATION TERMINAL APPARATUS AND PATIENT IMAGE DATA ACQUISITION METHOD

(71) Applicant: Olympus Corporation, Tokyo (JP)

(72) Inventors: Tetsuya TOYODA, Tokyo (JP); Masaru IKEDA, Fujimi-shi (JP); Hideaki YOSHIDA, Tokyo (JP); Osamu NONAKA, Sagamihara-shi (JP)

(21) Appl. No.: 16/151,326

(22) Filed: Oct. 3, 2018

(30) Foreign Application Priority Data
Dec. 19, 2017 (JP) 2017-242988

(51) Int. Cl. G16H 50/20; G16H 30/40; G16H 50/70; G16H 10/60; A61B 5/00

(52) U.S. Cl. CPC G16H 50/20; G16H 30/40; A61B 5/0013; G16H 10/60; G16H 50/70

(57) ABSTRACT

A medical support system for supporting medical service by sharing patient information includes: a file server including a patient information database, the file server being connected to a network; and an information terminal apparatus configured to communicate with the file server via the network and transmit inputted order information to the file server, and the file server creates corresponding shooting condition information based on the order information transmitted from the information terminal apparatus and transmits the shooting condition information and the order information to the information terminal apparatus.

### CROSS REFERENCE TO RELATED APPLICATION

This application claims the benefit of Japanese Application No. 2017-242988 filed in Japan on Dec. 19, 2017, the contents of which are incorporated herein by this reference.

### BACKGROUND OF THE INVENTION

### I. Field of the Invention

This invention relates to a medical support system for supporting medical service via patient information sharing, an information terminal apparatus that handles image data from among patient information, the information terminal apparatus being employed in the medical support system, and a patient image data acquisition method for acquiring image data as patient information.

### 2. DESCRIPTION OF RELATED ART

In recent years, a type of information terminal apparatus that can easily handle various types of information, has a communication function and is compact and portable has obtained popularity.

This type of information terminal apparatus is configured to be capable of performing direct intercommunication with another information terminal apparatus of the same type and exchanging various types of information with the other information terminal apparatus and also be capable of accessing, e.g., a file server located at a remote site via a network and transmitting/receiving various types of information to/from the file server.

Therefore, conventionally, network systems configured so as to handle and share various information utilizing information and communication technology (ICT) in which a plurality of information terminal apparatuses and a file server with various types of information accumulated are interconnected via an existing or dedicated network have been put into practical use and rapidly prevailing.

Examples of this type of network system include a medical support system built so that in, e.g., a medical institution, a health professional or the like (for example, a person provided with any of various healthcare-related qualifications such as a doctor, a nurse, any of various professional technicians or a certified care worker) accesses a file server installed in a medical base such as a hospital via an external network or an internal network (for example, a hospital information system (HIS)) using an information terminal apparatus and intercommunicates with the file server to enable sharing of healthcare-related information (for example, electronic medical record information or patient information).

Conventionally, for this type of medical support system, for example, Japanese Patent Application Laid-Open Publication No. 2006-195669 discloses various proposals.

The medical support systems disclosed by, e.g., Japanese Patent Application Laid-Open Publication No. 2006-195669 are medical support systems configured to perform, e.g., electronic medical record management, order management and task schedule management using information terminal apparatuses each used inside or outside a medical facility and having various functions such as a voice communication function, a data communication function and a personal authentication function.

However, this type of medical support system is supposed to be used for, for example, healthcare services such as an on-line examination, home-visit medical care, home-visit care for patients having difficulty in visiting a hospital. In this case, for example, use of the medical support system enables, e.g., patients having difficulty in moving around by themselves or patients living in remote areas away from, e.g., a hospital to easily receive, e.g., medical examination or consultation by a doctor without visiting a medical base such as a hospital.

Also, if it is possible that: a health professional such as a nurse or a certified care worker visiting a patient or, e.g., a care giver such as a patient family member or a close relative or the like at the patient' home acquires image information, as patient information, using a predetermined information terminal apparatus; the patient information (e.g., image information) thus acquired is transmitted to and accumulated in a file server installed at a medical base such as a hospital; and the patient information (image information) is shared with a doctor in charge of the patient (what is called “attending doctor”), the convenience of the doctor being able to perform a follow-up of the patient or grasp a current status of the patient by referring to the image without visiting the patient at home is conceivable. Such trend has created a need to make acquisition of patient information more open.

### SUMMARY OF THE INVENTION

A medical support system according to an aspect of the present invention is a medical support system for supporting medical service via patient information sharing, the system including: a file server including a patient information database, the file server being connected to a network; and an information terminal apparatus configured to communicate with the file server via the network and transmit inputted order information to the file server, and the file server creates corresponding shooting condition information based on the order information transmitted from the information terminal apparatus and transmits the shooting condition information and the order information to the information terminal apparatus.

An information terminal apparatus according to an aspect of the present invention includes: an image pickup section configured to acquire image data; a communication section configured to communicate with a file server via a network and transmit inputted order information to the file server; and an image pickup control section configured to receive shooting condition information created based on the order information from the file server, performs image shooting according to the shooting condition information and associates the order information with the acquired image data.

A patient image data acquisition method according to an aspect of the present invention is a method for acquiring patient image data included in patient information in a medical support system for supporting medical service via patient information sharing, the method including: creating order information in response to a doctor's order; creating shooting condition information according to the order information; transmitting the shooting condition information to an information terminal apparatus; and causing the information terminal apparatus to acquire corresponding image data in response to the shooting condition information. Accordingly, medical information, which has conventionally been acquired at a dedicated environment or facility upon an instruction being given by a doctor to a health professional or a patient, can be released from the conventional restrictions and acquired in a more open environment.

Benefits of this invention will be further clarified by the following detailed description.

### BRIEF DESCRIPTION OF THE DRAWINGS

FIG. 1 is a conceptual diagram illustrating a schematic configuration of a medical support system according to an embodiment of the present invention;

FIG. 2 is a conceptual diagram illustrating an overview of operation of the medical support system according to the embodiment of the present invention, depending on a situation of use (explanatory diagram of a first pattern);

FIG. 3 is a conceptual diagram illustrating an overview of operation of the medical support system according to the embodiment of the present invention, depending on a situation of use (explanatory diagram of a first pattern);

FIG. 4 is a conceptual diagram illustrating an overview of operation of the medical support system according to the embodiment of the present invention, depending on a situation of use (explanatory diagram of a second pattern);

FIG. 5 is a conceptual diagram illustrating an overview of operation of the medical support system according to the embodiment of the present invention, depending on a situation of use (explanatory diagram of second and third patterns);

FIG. 6 is an explanatory diagram of use after first to third patterns in operation of the medical support system according to the embodiment of the present invention;

FIG. 7 is an explanatory diagram of use after first to third patterns in operation of the medical support system according to the embodiment of the present invention;

FIG. 8 is a block configuration diagram illustrating a major configuration of an information terminal apparatus in the medical support system according to the embodiment of the present invention;

FIG. 9 is a block configuration diagram illustrating a major configuration of a file server in the medical support system according to the embodiment of the present invention;

FIG. 10 is a flowchart illustrating a part of operation of a second terminal from among information terminal apparatuses in the medical support system according to the embodiment of the present invention;

FIG. 11 is a flowchart illustrating another part of operation of the second terminal from among information terminal apparatuses in the medical support system according to the embodiment of the present invention;

FIG. 12 is a flowchart illustrating operation of a first terminal used by a doctor from among information terminal apparatuses in the medical support system according to the embodiment of the present invention; and

FIG. 13 is a flowchart illustrating operation of the file server in the medical support system according to the embodiment of the present invention.

### DETAILED DESCRIPTION OF THE PREFERRED EMBODIMENTS

The present invention will be described below based on the embodiment illustrated in the drawings. Each of the drawings used for the below description is a schematic one, and in order to illustrate respective components in sizes that are large enough to be recognized in the drawings, the components may be illustrated so as to be different in, e.g., scale and dimensional relationship between members. Therefore, the present invention is not limited to the illustrated forms in terms of, e.g., the number of the components, shapes of the components, ratios in size between the components and relative positional relationships between the respective components indicated in each drawing.

### Embodiment

A medical support system according to an embodiment of the present invention is a network system to be used in, e.g., a medical institution, and is an example of a network system for supporting medical service by sharing, e.g., patient information including, e.g., a medical image of a patient or a medical reference image.

Here, a medical image refers to image information formally approved as an image that in response to a doctor's order (request or instruction), a predetermined health professional or the like acquires based on a prescribed setting or a format.

Also, the health professional or the like refers to any of persons provided with various types of healthcare-related qualifications, including, e.g., a doctor, a nurse, various professional technicians and a certified care worker (what is called “healthcare staff”).

Therefore, a medical image refers to, in addition to an image acquired by a doctor himself/herself using predetermined medical equipment (for example, any of various image pickup apparatuses such as a medical endoscope apparatus or a medical ultrasound examination apparatus) according to his/her own order (e.g., a medical care policy) (for example, an endoscopic image, an ultrasound image or a picked-up image), one similar to an image that a health professional other than the doctor (e.g., a professional technician having a predetermined healthcare qualification (for example, an endoscopy technician, an ultrasound technician or a clinical radiologist)) acquires in response to the doctor's order.

Also, a medical reference image is referred to as image information acquired by a health professional other than a doctor, a professional technician or the like (healthcare staff having no particular professional qualification (for example, a certified care worker)) or a general person who is not a health professional (for example, a non-health professional such as a patient's family member or close relative, which is hereinafter referred to as “patient's family member”). In this case, the medical reference image is an image acquired according to prescribed shooting condition information set in response to a doctor's order (request or instruction), predetermined settings and a format. The medical reference image is not one approved as an official medical image but may be image information acknowledged by a doctor as a reference image for medical practice.

Examples of a doctor's order (e.g., a request or an instruction) are indicated below.

In other words, in normal cases, e.g., after examination of a patient that is a subject of, e.g., treatment, examination or test (hereinafter referred to as “subject patient”), a doctor accesses a file server installed in a medical base, which is a hospital or the like, via an internal network (in-house network) using an in-house terminal (for example, an internal terminal apparatus such as a PC is installed in a consultation room in the hospital or the like), and puts a record of the examination of the day into an electronic medical record file for the subject patient in an electronic medical record database accumulated in the file server.

At that time, the doctor may concurrently give an instruction for, e.g., a next test. For that purpose, the doctor opens a predetermined order screen using the in-house terminal and creates order information for, e.g., a desired test from the order screen.

Order information provides, in addition to, for example, an instruction for selection of a desired shooting item from among a plurality of shooting types (for example, X-ray photography, CT, MRI, endoscopy, ultrasound scanning and general shooting) and an instruction for selection of a desired shooting object region (for example, the breast region, the abdominal region, the head region or an elbow joint), for example, a specific shooting instruction for selection of, e.g., the front side and a lateral side of the left knee. Furthermore, if a more detailed instruction is to be provided as necessary, for example, such detailed instruction is stated in, for example, a comment column.

As described above, many of general medical images are each resulting from a doctor contemplating and designating an image and immediately afterward providing an instruction to a health professional and a patient to acquire such image specifically; however, in the present application, some ingenuity is taken to enable shooting an image contemplated and designated by a doctor without such constraints. Here, examples of the detailed instruction are as follows.

Examples of a doctor's order as a detailed instruction where the order is “an order for shooting an image for observing the course of a wound of a predetermined subject patient” include:

(1) Shooting in a macro mode (shooting scaling factor: 1:2); and
(2) Shooting the image with a predetermined color chart included in order to determine the color of the skin.

Also, not only an instruction for shooting a still image, but also, for example, an instruction for shooting a movie or an instruction for shooting a still image or movie with voice is conceivable.

The order information thus created is associated with patient information on the subject patient and then transferred to a terminal apparatus used by, e.g., a health professional in a relevant department that handles tests.

Here, order information in a medical institution is information including, e.g., “when”, “where”, “who”, “to whom”, “how”, “what apparatus to be used”, and “what test to be conducted”. Such information may be converted into data as text information in a specific language, may be replaced with signs or flags which are recorded for each of specific data regions and which can be interpreted by using a table or the like, or may be recorded digitally as a data set that can be interpreted according to a specific law. As the above-described standard information, information on apparatuses, medical materials, medicines, and test codes, the formats specified in DICOM (Digital Imaging and Communications in Medicine) and the codes simplified by a text may be used. Also regarding the items that are not specified in DICOM, according to the standards of Health Level Seven (HL7) and other medical information and communication technology (ICT), versatility of the information can be ensured.

In other words, in a hospital, “who” corresponds to, e.g., a medical examination department or a person in charge of tests; however, in the present invention, “who” is a user who shoots a patient image via, e.g., an information terminal apparatus.

“To whom” corresponds to various types of information included in medical records, for example, a patient's basic attribute information such as date of birth, gender and blood type, and a patient ID. Such information is to be supplied by, e.g., an in-house system or a doctor, but in consideration of, e.g., handling of personal information, only necessary items of information may be included in order information.

“What apparatus to be used” corresponds to, e.g., a CT apparatus, an MRI apparatus, an endoscope or an image pickup apparatus (camera).

“What test to be conducted” is information on, e.g., the number of photos of a region (e.g., the breast or the foot) of a patient to be shot and an intensity of radiation. In the present invention, “what test to be conducted” corresponds to, e.g., shooting parameters such as a composition, a distance, a focal length, a shooting magnification, exposure, a focus and an aperture when an image is shot using an information terminal apparatus, image quality, an image size and a file format. Furthermore, e.g., the number of images to be shot and/or settings for the shooting parameters may be included. These pieces of information may be included information on “what apparatus to be used”.

As described above, order information includes various types of information, and may further include, e.g., a shooting procedure for reminding shooting while providing, e.g., an indication to shoot images in sequence according to, e.g., ordering list information in which orders are collected as a list, so as to enable a test or treatment to be performed. In this case, advantages are provided in preventing missing some images, or in efficiency.

In consideration of such advantages, these pieces of information (patient basic attribute information, etc.) may be referred to as, e.g., “supplementary information for management”, “supplementary information for medical examination” or “medical examination management information”.

For example, even in the case of a same patient, the patient may have different types of symptoms such as diseases or injuries (may be subjected to different studies), and tests in different situations (including shooting) may be expected for the respective symptoms. Therefore, it is also necessary to distinguish these tests. For example, it is conceivable that where a medical checkup is conducted every year, different pieces of study information are handled for the respective years.

As a matter of course, a shooting method can be changed according to the situation, and in that case, if a result of shooting being performed is not the same as a result expected by an order, information on the performance may be added as tag information for the relevant image.

In the below-described embodiment, the above-described pieces of information are collectively referred as order information, but all of such pieces of information are not essential but only a part of such pieces of information may be included.

Outside medical facilities, main use cases are home-visit medical care or home medical care. In that case, an act of, e.g., shooting an image of a patient at home is performed. For support of home medical care, under an environment in which no sufficient medical equipment can be provided, it is important to enable use of such information when an image is shot using, e.g., an image pickup function of a camera or a smartphone, which is a general consumer product that is not dedicated medical equipment. Therefore, a situation in which a health professional such as a nurse or a doctor performs shooting using an apparatus that has received a signal as an order recipient when he/she visits a patient at home may be contemplated.

Order information thus created by a doctor is a group of pieces of information in which a plurality of instructions relating to medical practice are collectively included.

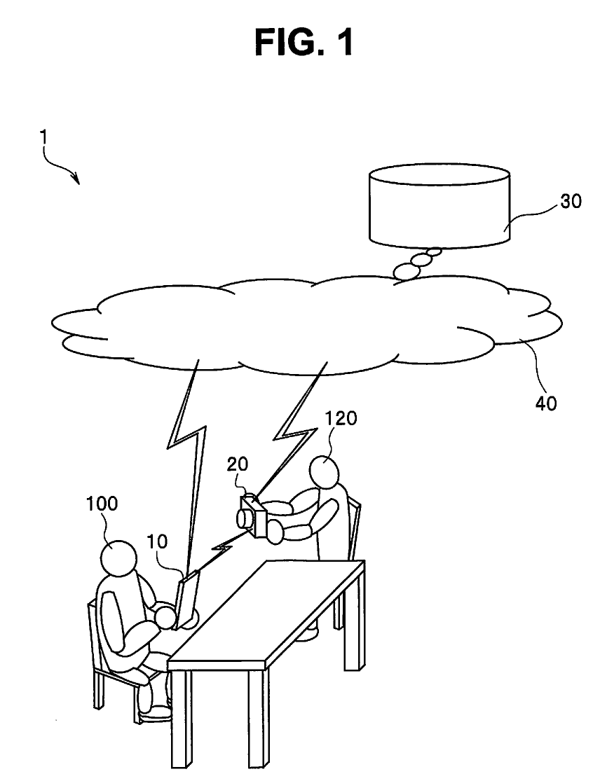

A concept of a medical support system according to an embodiment of the present invention will be described below with reference to FIG. 1. FIG. 1 is a conceptual diagram illustrating a schematic configuration of a medical support system according to an embodiment of the present invention.

As illustrated in FIG. 1, a medical support system 1 according to the present embodiment mainly includes, e.g., a plurality of information terminal apparatuses 10, 20, a file server 30 and a network 40 provided to connect the plurality of information terminal apparatuses 10, 20 and the file server 30 with each other.

In this case, the plurality of information terminal apparatuses 10, 20 can directly communicate with each other and each can communicate with the file server 30 via the network 40.

As means for direct communication between the plurality of information terminal apparatuses 10, 20, for example, existing short-distance communication means is employed.

Also, for communication between the plurality of information terminal apparatuses 10, 20 and the file server 30, for example, existing general communication means for communication via an external network such as the Internet, the communication means being connected via an internal network (a wireless LAN or a wired LAN), is employed.

Each of the information terminal apparatuses 10, 20 is a compact and portable electronic device having an image pickup function capable of acquiring image information and also having, in addition a function of direct communication with another information terminal apparatus, a function of communication via the network 40.

As specific forms of the information terminal apparatuses 10, 20, for example, a communication function-equipped image pickup apparatus (camera) and a smartphone and a tablet PC, which are image pickup function-equipped communication apparatuses, are contemplated.

In the example illustrated in FIG. 1, from among the plurality of information terminal apparatuses 10, 20, the information terminal apparatus indicated by reference numeral 10 is an information terminal apparatus used by a doctor 100. In the below description, the information terminal apparatus is referred to as “first terminal 10”. Also, in FIG. 1, as an example, the first terminal 10 is illustrated as one having a form of a tablet PC.

On the other hand, from among the plurality of information terminal apparatuses 10, 20, the information terminal apparatus indicated by reference numeral 20 is an information terminal apparatus used by a person 120 (for example, a patient's family member) other than the doctor 100. In the below description, the information terminal apparatus is referred to as “second terminal 20”. Also, in FIG. 1, as an example, the second terminal 20 is illustrated as having a form of a camera, which is a general image pickup apparatus.

Here, the plurality of information terminal apparatuses 10, 20 may each have a table PC form or a camera form, or another form, for example, a smartphone form, and basically have similar functions irrespective of the forms.

In other words, for each of information terminal apparatuses (10, 20) that can be employed for the medical support system 1 according to the present embodiment, any information terminal apparatus can be employed equally irrespective of the form of the apparatus as long as the information terminal apparatus has at least an image pickup function and a communication function. Therefore, the forms of the respective information terminal apparatuses 10, 20 illustrated in FIG. 1 are mere examples.

An overview of operation of the medical support system 1 according to the present embodiment configured as above is as follows.

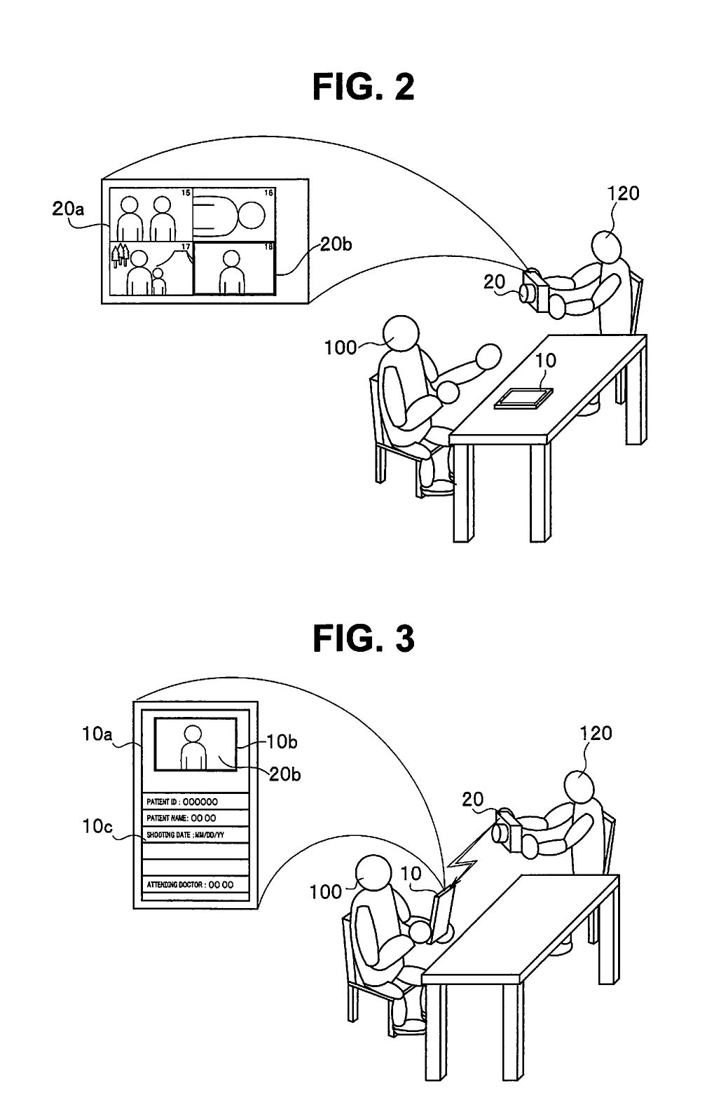

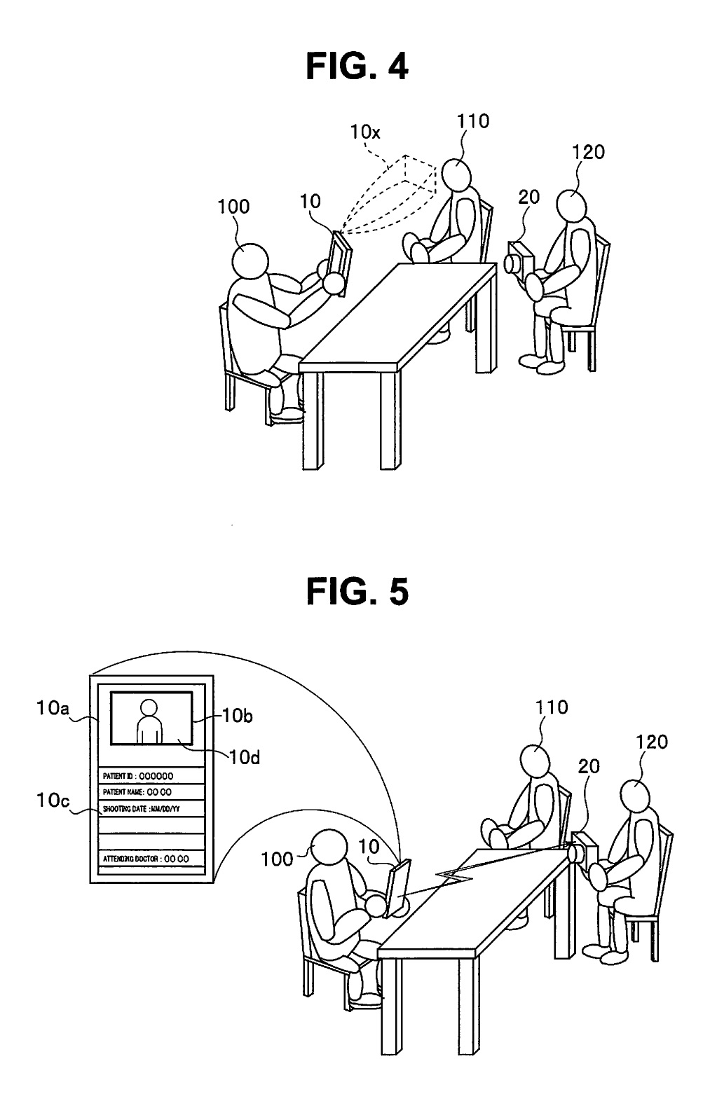

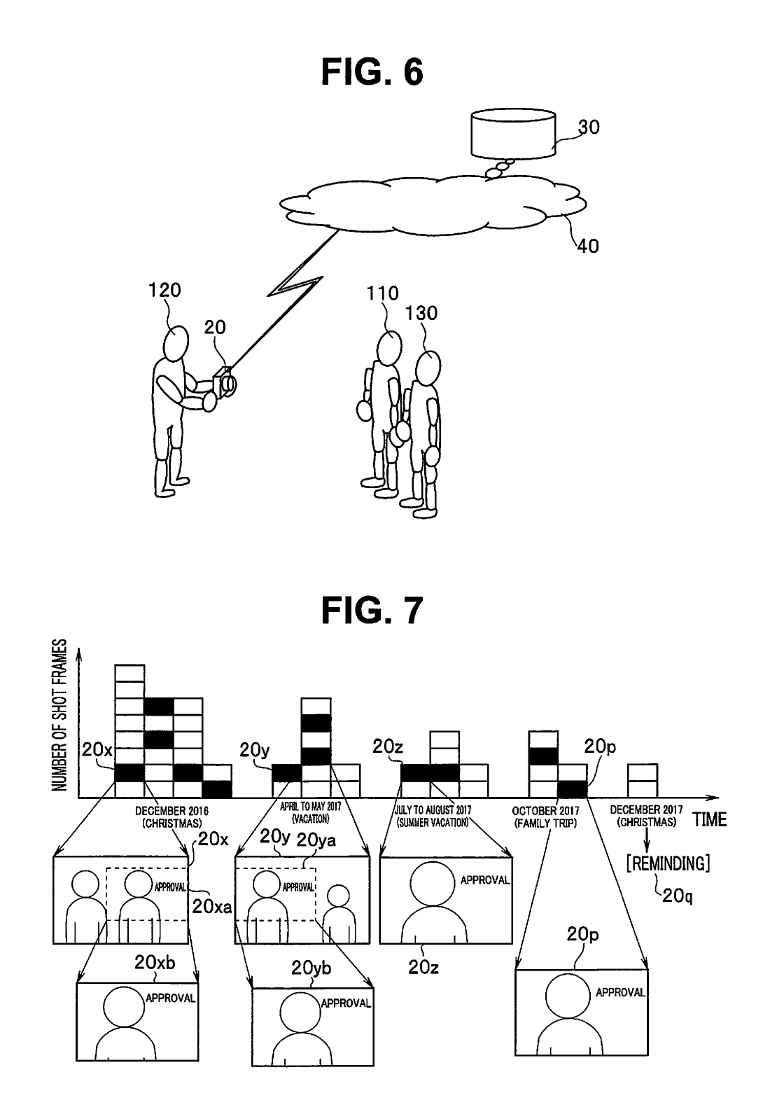

FIGS. 2 to 7 are conceptual diagrams illustrating an overview of operation of the medical support system according to the present embodiment depending on respective situations of use. From among the figures, FIGS. 2 and 3 are conceptual diagrams illustrating a first pattern of operation of the medical support system according to the present embodiment. FIGS. 4 and 5 are conceptual diagrams illustrating a second pattern of operation of the medical support system according to the present embodiment. FIG. 5 doubles as a conceptual diagram illustrating a third pattern of operation of the medical support system according to the present embodiment. FIGS. 6 and 7 are conceptual diagrams illustrating use after reception of a sample image (standard image) for a medical reference image from a doctor, depending on the respective situations of use described as the first to third patterns.

Operation of the medical support system 1 according to the present embodiment will briefly be described for each of specific situations of use (use patterns).

First, a first pattern in a situation of use of the medical support system 1 according to the present embodiment is as follows (see FIGS. 2 and 3).

In other words, the situation of use illustrated in FIG. 2 indicates a situation in which a patient's family member 120 of a certain patient visits a consultation room of a doctor 100, which is an attending doctor for the patient, in a medical base such as a hospital to which the doctor 100 belongs (situation of what is called proxy examination).

Here, it is contemplated that not the patient himself/herself, but the patient's family member 120 visits the hospital or the like because, for example, the patient having difficulty in visiting the hospital or the like for a reason that, e.g., the patient having difficulty in moving around by himself/herself or lives in an area distant from the hospital or the like.

In this case, the patient's family member 120 brings a second terminal 20 (camera) with him/her, the second terminal 20 being an information terminal apparatus with a plurality of pieces of image information (image data) recorded, the plurality of pieces of image information including images shot of the patient, which is not present there, at home.

The patient's family member 120 shows the doctor 100 the plurality of pieces of image information recorded in the second terminal 20 (image list display indicated by reference numeral 20a in FIG. 2).

The doctor 100 selects an image that he/she deems as an image that can be employed as a medical reference image that serves as patient information, from among the plurality of images recorded in the second terminal 20 brought by the patient's family member 120 (image within the thick frame border indicated by the reference numeral 20b in FIG. 2).

Here, the doctor 100 performs work for approving the selected image 20b as a medical reference image and employing the selected image 20b as a sample image (standard image) for future medical reference images.

In other words, at this point of time (under a situation in which the patient's family member 120 merely shows the doctor 100 the images in the second terminal 20), the selected image 20b is a mere general image privately acquired by the second terminal 20 privately owned by the patient's family member 120.

In order to make such general image be a medical reference image, the doctor 100 provides predetermined medical information to an image file of the selected image 20b as meta data to approve the selected image 20b as a medical reference image. In the below description, an example of a procedure of the approval work will briefly be described.

Note that an image data file handled in the medical support system 1 according to the present embodiment includes predetermined meta data including, for example, medical information, separately from meta data included in a conventional general image data file (for example, Exif (exchangeable image file format) data); and the predetermined meta data is specifically referred to as “medical meta data”. The medical meta data may be provided in a form of a file that is separately from the conventional general meta data or may be provided in a form integrated with the conventional general meta data. However, regardless of which form to be employed, it is important that image data and medical meta data are associated with each other. As a result of image data and medical meta data being associated with each other, even if the image data and the medical meta data are included in respective independent files, when an operation for image data (for example, operation of moving or copying the image data to another medium or deleting the image data) is performed, a predetermined operation is necessarily performed together with the associated meta data. Therefore, for example, the medical meta data is prevented from existing alone.

In order to provide the medical meta data to a predetermined image data file, first, the doctor 100 has the selected image 20b transferred from the second terminal 20 at the patient's family member 120 to the first terminal 10 at the doctor 100 (see FIG. 3).

Upon receipt of the selected image 20b, as illustrated in FIG. 3, the doctor 100 performs the work for providing predetermined medical meta data to the received selected image 20b, using the first terminal 10.

Here, reference numeral 10a in FIG. 3 indicates an example of a display screen of the first terminal 10. Here, the reference numeral 10b in FIG. 3 indicates a display frame of the selected image 20b after the transfer of the selected image 20b to the first terminal 10. Reference numeral 10c in FIG. 3 indicates a detail display column for medical meta data the doctor adds to the selected image 20b.

The doctor 100 inputs desired information to the display column 10c by performing a predetermined operation of the first terminal 10.

In addition to the medical meta data added to the selected image 20b via the input by the doctor 100 as described above, items overlapping items in the existing meta data previously provided to the selected image 20b, for example, shooting time/date information, shooting device information (e.g., information unique to the camera) are automatically provided and displayed in the display column 10c.

Also, if the doctor 100 does not handle patient information including image information on the subject patient of this time using the first terminal 10 for the first time but has used the first terminal 10 for the subject patient before, the first terminal 10 should already include patient information on the subject patient.

Therefore, when the patient image (selected image 20b) has been transferred from the second terminal 20 as described above, corresponding patient information (for example, a patient ID) can automatically be detected by, for example, subjecting the image data of the selected image 20b to face detection processing or detecting the meta data of the selected image 20b. Therefore, the first terminal 10 can automatically incorporate desired information as medical meta data for the selected image 20b using the detected patient information.

As a result of the predetermined medical meta data being provided to the selected image 20b as described above, the selected image 20b is approved as a medical reference image by the doctor 100.

Upon end of such work, the doctor 100 sends the medical meta data-provided selected image 20b back to the second terminal 20.

Upon receipt of the selected image 20b, the second terminal 20 stores the medical meta data-provided selected image 20b with a different name so that the image file of the original selected image 20b remains. As a matter of course, settings may be made so that a form of the storing in this case is an overwrite save.

Also, simultaneously with the above sending, the first terminal 10 may transfer the medical meta data-provided selected image 20b to the file server 30 (not illustrated in FIGS. 2 and 3; see FIG. 1).

In this case, examples of the medical meta data provided to the selected image 20b by the first terminal 10 include, e.g., a patient ID (patient identification information), information relating to doctors in charge including, e.g., the attending doctor and/or the medical institution, information of the approval as a medical reference image, and treatment schedule information. Additionally, the medical meta data includes, e.g., various types of shooting condition information for acquiring an image similar to the selected image 20b (e.g., setting information such as various shooting parameters for enabling an equal and similar image to be acquired each time shooting is performed) and/or a thumbnail image of a sample image. Examples of the shooting condition information include, e.g., various types of image pickup-related information such as exposure information, white balance information, sensitivity information, optical system focal length information, information on a distance to an object, a shooting scale factor information and angle information.

Additionally, the medical meta data may further include various types of information included in, e.g., Exif image data used as a conventional general image file (for example, a shooting location (GPS information), time/date information and shooting condition information).

Although the above description has been given in terms of a case where various types of information related to shooting (shooting condition information) are included in the medical meta data, but the present embodiment is not limited to this case, and a data file of the shooting condition information may be created separately from the image data file. Then, the data file of the shooting condition information is recorded in association with the corresponding image data file. Then, when the image data is transferred to the second terminal 20, the related shooting condition information data file is also transferred together with the image data to the second terminal 20.

Consequently, the selected image 20b approved as a medical reference image by the doctor 100 is recorded in the second terminal 20. Subsequently, the patient's family member 120 periodically shooting an image of the subject patient, e.g., at home using the second terminal 20. The image periodically shot in such situation is referred to as, for example, “watching-over image”. The watching-over image can be obtained by an action of the patient's family member 120 shooting an image of the subject patient using the second terminal 20, but at that time, the patient's family member 120 may perform normal shooting with no particular regard to shooting a medical reference image.

The second terminal 20 can detect that the subject patient is a shooting object, by using, e.g., a face detection technique. Therefore, upon detection of the subject patient, the second terminal 20 automatically sets the shooting parameters (e.g., the exposure setting, the white balance setting, the sensitivity setting and the focal length setting) corresponding to a shooting operation mode for acquiring a new medical reference image (hereinafter referred to as “watching-over image shooting mode”) with reference to the shooting condition information accompanying the medical reference image (selected image 20b) approved by the doctor. Furthermore, at this time, guide display (assist display) of shooting condition information such as a composition, a shooting angle and a distance to the subject automatically appear on a display section (for example, displayed so as to overlap with a live view image).

New image data acquired as described above is provided with meta data corresponding to the medical meta data accompanying the medical reference image (selected image 20b) approved by the doctor and is recorded as a new medical reference image or an image candidate for a new medical reference image.

Consequently, the patient's family member 120 can easily acquire a new medical reference image according to the doctor 100's order, using the second terminal 20.

Also, in the above-described example, detection of the subject patient by means of face recognition is performed each time shooting is performed, but another method of shooting may be employed as follows.

For example, the patient's family member 120 performs normal shooting daily using the second terminal 20. In such case, the subject patient is often not necessarily included in a plurality of images acquired by the normal shooting using the plurality of images second terminal 20.

Therefore, the plurality of images may be subjected to face detection processing at a predetermined timing arbitrarily by a user or automatically to detect images including the subject patient.

Then, if image data similar to the medical reference image (selected image 20b) approved by the doctor is found from among the images including the subject patient, predetermined medical meta data may be provided to the similar image and the similar image may be recorded as a new medical reference image or an image candidate for a new medical reference image.

Consequently, a plurality of image data are acquired as new medical reference images by the second terminal 20. Then, when the patient's family member 120 brings the second terminal 20 with him/her to the hospital or the like next time he/she visits the hospital or the like (at the time of next proxy examination), the plurality of image data (medical meta data-provided image data only) is automatically transferred from the second terminal 20 to the file server 30 installed in the medical base such as the hospital or the like. Alternatively, the doctor 100 may browse the second terminal 20 and selectively transfer the plurality of image data to the file server 30 via, e.g., a manual operation.

Although the above description has been given taking the patient's family member as an example of a person other than a health professional, but the present embodiment is not limited to such example, and, for example, may be applied to, for example, a doctor's visit at home or home-visit care. In such case, e.g., a nurse or a certified care worker who visits a patient's home serves as a person who operates a second terminal 20.

Consequently, patient image data acquired by, e.g., a health professional other than a doctor or a patient's family member that is not a health professional can be handled as a medical reference image that can be approved by the doctor.

The above is an overview of the first pattern of use of the medical support system 1 according to the present embodiment.

Next, a second pattern of use of the medical support system 1 according to the present embodiment is as follows (see FIGS. 4 and 5).

In other words, the situation illustrated in FIG. 4 indicates a situation in which a subject patient 110 and a patient's family member 120 accompanying the patient 110 visit a consultation room in a hospital or the like (medical base) that a doctor 100 who is an attending doctor for a patient 110, belongs to.

Here, it is assumed that the patient himself/herself having some difficulty in moving around by himself/herself and visits the hospital or the like with his/her family member because the patient has difficulty in frequently visiting the hospital or the like for a reason that, e.g., the patient lives in an area distant from the hospital or the like.

Here, a situation in which the patient himself/herself visits the hospital is assumed, but the present embodiment is not limited to such situation and may be similarly applied to, for example, a situation in which a doctor visits a patient at home.

In this case, a patient's family member 120 brings a second terminal 20 (camera) with him/her, the second terminal 20 (camera) being an information terminal apparatus capable of acquiring a plurality of pieces of image information (image data).

Under such situation, the doctor 100 first shoots an image of the patient 110 using a first terminal 10 according to predetermined settings and a format so that the image can be used as a medical image.

The example illustrated in FIG. 4 indicates a case where the upper half of the body of the patient, the upper half including the face, is a shooting subject. Dotted line 10x in FIG. 4 conceptually indicates an overview of a shooting range when the patient 110 is a shooting subject.

In this case, the doctor 100 shoots an image that can be employed as a medical image that serves as patient information. An image of the subject patient shot as described (hereinafter referred to as “patient image”) is provided to the patient's family member 120 as a sample image (standard image) for future medical reference images.

Therefore, first, the doctor 100 performs, e.g., an operation of providing predetermined medical meta data to the patient image and an operation of approving the patient image as a medical image using the first terminal 10, using the first terminal 10 (see FIG. 5).

Here, FIG. 5 illustrates a state in which a patient image 10d acquired by the first terminal 10 is displayed. Reference numeral 10a in FIG. 5 denotes a display screen of the first terminal 10. Reference numeral 10b in FIG. 5 denotes a display frame of the patient image 10d acquired by the first terminal 10. Reference numeral 10c in FIG. 5 denotes a display column for medical meta data to be added. Here, details of the medical meta data are similar to the details in the above-described first pattern.

The medical meta data and the medical image approval information may automatically be provided to the patient image 10d at the time of shooting. The patient image 10d is an image shot by the doctor 100 himself/herself using his/her personal first terminal 10. If the doctor 100 having a past record in handling patient information including the subject patient of this time using the first terminal 10, the patient information on the subject patient must be included in the first terminal 10. Therefore, as described above, at a point of time of an image of a patient being shot using the first terminal 10, for example, corresponding patient information (for example, a patient ID) can automatically be detected by means of, e.g., face detection processing, and desired information can automatically be incorporated into medical meta data using the patient information. Also, since the first terminal 10 is used solely by the doctor 100, the image can naturally be approved as a medical image.

Consequently, the patient image 10d is provided with predetermined medical meta data and approved as a medical image by the doctor 100. After end of such work, the doctor 100 transfers the medical meta data-provided patient image 10d to the second terminal 20 of the patient's family member 120. At this time, as with the first pattern described above, e.g., shooting condition information is transferred together with the patient image 10d.

Then, the patient image 10d, which is a medical image approved by the doctor 100, is recorded in the second terminal 20. Afterward, the patient's family member 120 shoots an image of the patient, e.g., at home using the second terminal 20 (watching-over image shooting). Subsequent operation is similar to the operation in the first pattern of use described above.

Also, a new patient image acquired using the second terminal 20 by the patient's family member 120 according to the medical image approved by the doctor is an image shot by the patient's family member 120, and thus, medical meta data, which is medical reference image, is provided to the image.

The above is an overview of the second pattern of use of the medical support system 1 according to the present embodiment.

In the above-described second pattern of use, a patient image shot by the doctor 100 using the first terminal 10 is set as a medical image and is transferred as a sample image to the second terminal 20 after provision of medical meta data to the patient image; however, the present embodiment is not limited to this form.

For example, as a third pattern of use, the following situation is conceivable (see FIG. 5).

First, it is assumed that a database of a plurality of sample images according to various symptoms, affected parts, etc., is built in a medical base such as a hospital and provided in the file server 30 in advance.

A doctor 100 accesses the file server 30 using a first terminal 10 as necessary, acquires a desired sample image and provides the sample image to a patient's family member 120.

In this case, also, the patient's family member 120 can easily acquire a medical reference image or an image candidate for a medical reference image merely by shooting an image of the patient using a second terminal 20.

After receiving a sample image for a medical reference image from the doctor 100 in such a manner described above via the second terminal 20, the patient's family member 120 can automatically acquire a medical reference image or an image candidate for a medical reference image by shooting an image of the patient using the second terminal 20.

In this case, when the patient's family member 120 shoots a patient image using the second terminal 20, the patient's family member 120 can automatically acquire a medical reference image similar to the sample image or an image candidate for such medical reference image merely by using the second terminal 20 ordinarily without having a particular consciousness of shooting a patient image for a medical purpose.

FIG. 6 indicates a situation in which a patient's family member 120 ordinarily uses a second terminal 20, and, for example, take photos including a patient 110 and another family member 130. A plurality of image data thus acquired are recorded and stored in chronological order in a storage medium (not illustrated) in the second terminal 20 (see FIG. 7). Also, at this time, it is conceivable that as necessary, predetermined image data is transferred to the file server 30 via the network 40. FIG. 7 conceptually illustrates a state of accumulation of a plurality of image data recorded in the second terminal 20.

As illustrated in FIG. 7, in the storage medium (not illustrated) in the second terminal 20, for example, folders are created by date or event, and in each folder, a plurality of relevant image data are recorded.

In the example illustrated in FIG. 7, date information and event name information such as “December, 2016 (Christmas)”, “April to May, 2017 (Vacation)”, “July to August, 2017 (Summer Vacation)”, “October, 2017 (Family Trip)” and “December, 2017 (Christmas)” are indicated. Thus, a state in which the plurality of images shot in the respective events are recorded in a folder-by-folder basis is indicated.

In this case, each cell indicates a frame of a shot image. Furthermore, blacked-out cells from among the frames of shot images each clearly indicate frames of images including the subject patient.

Here, detection of the subject patient in the images is performed by means of, e.g., face detection processing, for example, with reference to, e.g., medical meta data included in the above-described sample image or an image of a face registered in advance.

Then, the second terminal 20 searches for images similar to the sample image from among the plurality of images including the subject patient (blacked-out cells) at a predetermined timing. For this search processing, for example, processing such as pattern matching may be performed. In this case, not only a shot image itself, an entire area which is similar to the sample image, but also a shot image, a part of which is similar to the sample image, may be used as a medical reference image.

For example, image data indicated by reference numerals 20z, 20p in FIG. 7 are candidate image examples in which shot images themselves are similar to the sample image and can be approved as medical reference images as they are. As described above, a shooter can perform shooting according to a doctor's request without having a particular consciousness, and thus can acquire important information in daily life without having no technical knowledge. Also, it can be determined that the shooter is in a situation in which an image similar to the sample image can be shot even though the image is not a candidate image itself, and thus, it is possible to urge shooting an image similar to the sample image via, for example, a guide indication or a warning.

On the other hand, image data indicated by reference numerals 20x, 20y in FIG. 7 are examples of a case where a partial area of a shot image is similar to the sample image. In this case, example candidate images that can be approved as medical reference images, the images being obtained by cutting out respective partial areas of the images 20x, 20y and thus creating new image data, are indicated.

More specifically, for example, the patient's family member 120 shoots family photos including the patient using the second terminal 20 at the time of events in Christmas, Vacations, etc., (normal use).

Here, for example, it is assumed that the patient's family member 120 shoots photos including the patient 110 and the other family member 130 using the second terminal 20 in the situation illustrated in FIG. 6. It is also assumed that the images acquired as a result of shooting are the images indicated by reference numerals 20x, 20y in FIG. 7. From the images 20x, 20y, medical reference images similar to the sample image or images candidate for medical reference images can be created by trimming (cutting out) respective partial areas including the patient 110 (areas indicated by dotted frames in FIG. 7: reference numerals 20xa, 20xb) and subjecting the resulting images to enlargement processing.

The image frames illustrated in FIG. 7 are added with the statement “Approval”. This is an example of a display icon indicating that the relevant image is added with medical meta data and is image data that can be approved as a medical reference image.

Then, as illustrated in FIG. 7, where patient images are created as medical reference images at predetermined time intervals and these image data are transferred to the file server 30 and shared with the doctor, the doctor can use these images as a reference for grasping a status of the patient without seeing the patient face to face.

In other words, even where the patient himself/herself cannot go to see the doctor periodically, the doctor can see temporal change of a symptom of the patient without visiting the patient at home periodically.

In normal use of the second terminal 20, after a lapse of the predetermined time interval, neither an image similar to the sample image is provided nor an image including the subject patient is shot, the second terminal 20 indicates, e.g., a warning message reminding shooting a patient image on, e.g., a display screen (see reference numeral 20q in FIG. 7).

Consequently, the patient's family member 120 successively shoots patient images using the second terminal 20 at the predetermined time intervals. In this case, it is possible to continuously acquire medical reference images of a form that can be approved by the doctor, in mere normal use with no particular consciousness of the images being medical reference images.

The warning message of the reminding may be indicated at either of the time of operation of the second terminal 20 in a shooting mode and the time of operation of the second terminal 20 in a reproduction mode. For example, a warning message of reminding is indicated at the time of, e.g., startup of the second terminal 20 regardless of which operation mode is set.

Although in the situations of use described with reference to FIGS. 2 to 7, as examples of medical images and medical reference images, images each including the face of the patient are indicated, the present embodiment is not limited to this case. For medical images and medical reference images, for example, an image of, e.g., a diseased part of a patient, the diseased part differing from patient to patient or an image of a part in the vicinity of the diseased part after surgery is conceivable in addition to images of the face of the patient.

In order to achieve such operation as above, major configurations of an information terminal apparatus (first terminal 10 or second terminal 20) and the file server 30 in the medical support system 1 according to the present embodiment will be described below.

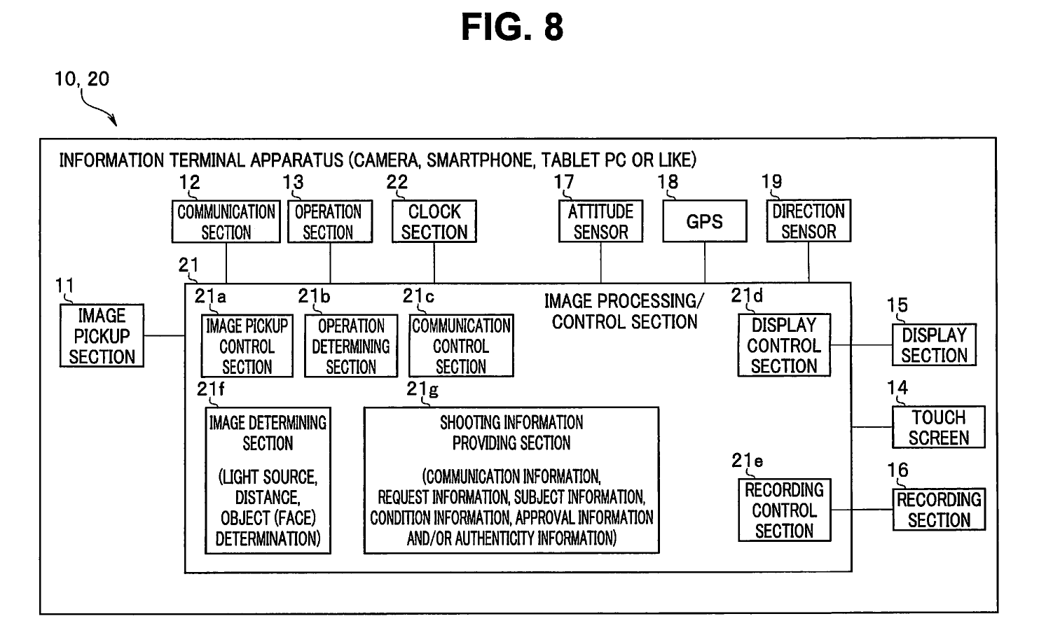

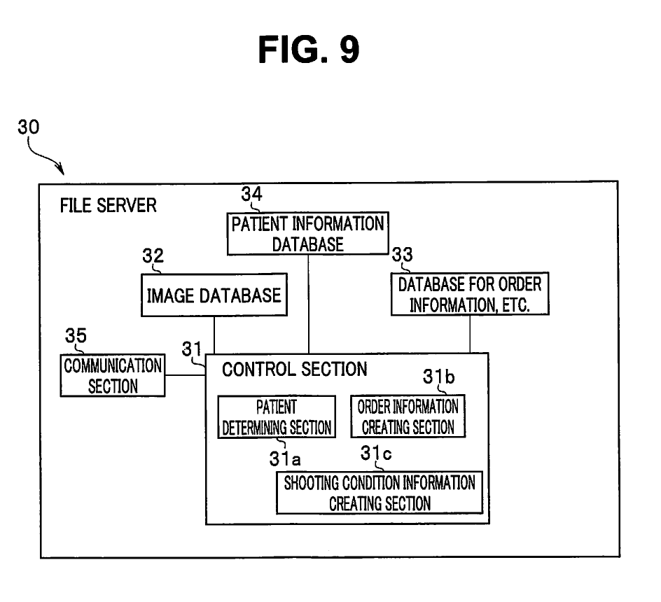

FIG. 8 is a block configuration diagram illustrating a major configuration of an information terminal apparatus in the medical support system according to the present embodiment. FIG. 9 is a block configuration diagram illustrating a major configuration of a file server in the medical support system according to the present embodiment.

First, a major configuration of an information terminal apparatus (10, 20) in the medical support system 1 according to the present embodiment will be described. As described above, the information terminal apparatuses include a first terminal 10 used by the doctor and a second terminal 20 used by a person other than the doctor (for example, a health professional other than the doctor or a patient's family member). These apparatuses are substantially the same in major configuration even if the apparatuses are different in form. Therefore, description will be given on the assumption that the first terminal 10 and the second terminal 20 have a same configuration.

As illustrated in FIG. 8, the information terminal apparatus (10, 20) mainly includes, e.g., an image pickup section 11, a communication section 12, an operation section 13, a touch screen 14, a display section 15, a recording section 16, an attitude sensor 17, a GPS 18, a direction sensor 19, an image processing/control section 21 and a clock section 22. In addition to the components stated here, the information terminal apparatus (10, 20) include various components. However, such components are parts that are not directly related to the spirit of the present invention, and thus illustration and description of such components are omitted. Various components, illustration and description of which are omitted here, are similar to respective components employed in a conventional information terminal apparatus having a general form.

The image pickup section 11 is a component unit that provides an image pickup function, the component unit including, e.g., an optical system configured to form an optical image, and a circuit including a photoelectric conversion element configured to convert an optical object image formed by the optical system into an electric signal. For a specific configuration of the image pickup section 11, a configuration that is substantially similar to a configuration of a conventional image pickup unit having a general form is employed. The image pickup section 11 is controlled by an image pickup control section 21a described later.

The communication section 12 is a component unit including, e.g., a circuit unit configured to enable communication with another device (for example, short-distance wireless communication means), and a circuit unit configured to enable communication with the file server 30 via the network 40. The communication means included in the communication section 12 is not limited to wireless communication means and may be wired communication means. The communication section 12 is controlled by a communication control section 21c described later.

The operation section 13 is a component unit including an operation member and a circuit section for in response to various operations performed by a user, generating various corresponding instruction signals.

The touch screen 14 is an operation member disposed in a form in which the touch screen 14 is superimposed on a display screen of the display section 15. The touch screen 14 is an operation member configured to in response to instructions provided by the user through operations with, e.g., his/her finger, generate various instruction signals. Although the touch screen 14 is included in the operation section 13, the touch screen 14 is one of operation members frequently employed in various electronic apparatuses in recent years and is a particularly important component and is thus indicated separately from the operation section 13.

For the operation section 13 and the touch screen 14, which operation member has been operated is determined by an operation determining section 21b described later. Based on a result of the determination, the image processing/control section 21 outputs a corresponding predetermined operation signal to a corresponding component unit.

The display section 15 is a component unit including, e.g., a display panel and a drive circuit for the display panel (not illustrated), the component unit being configured to provide a display function. On the display panel of the display section 15, an image based on image data acquired using the image pickup section 11 or a menu image generated by, e.g., various setting programs is displayed. The display section 15 is controlled by a display control section 21d.

The recording section 16 is a component unit for recording various data files. Examples of the data files recorded in the recording section 16 include, e.g., image information including image data and meta data, setting information in which various settings such as settings particular to setting items necessary for performing predetermined shooting are collected in the form of a file (more specifically, for example, setting information in which various settings to be made when a medical reference image is acquired are collected) and apparatus information on the relevant terminal apparatus (10, 20) or an apparatus on the other side of communication (e.g., a terminal or a file server) and various other information.

The attitude sensor 17, the GPS 18 and the direction sensor 19 include various sensor elements each configured to detect a state of the information terminal apparatus (10, 20), and circuit sections for the sensor elements, respectively. These circuit sections are similar to component units in practical use in a conventional terminal apparatus. Therefore, detailed descriptions of these components will be omitted.

The clock section 22 is an internal clock circuit that is what is called “real-time clock (RTC). The clock section 22 is a circuit section is used for, for example, providing, e.g., date information and time information to, e.g., an image data file, timing an interval between predetermined instruction timings during control processing, or temporal control.

The image processing/control section 21 includes a control circuit configured to perform overall control of the relevant information terminal apparatus (10, 20) and an image processing circuit configured to perform various types of image processing based on various types of image data.

The image processing/control section 21 includes, e.g., the image pickup control section 21a, the operation determining section 21b, the communication control section 21c, the display control section 21d, a recording control section 21e, an image determining section 21f and a shooting information providing section 21g.

The image pickup control section 21a is a circuit section or program software configured to control the image pickup section 11 to acquire data for live view image display or in response to a predetermined operation, perform an image pickup operation to acquire still image data or movie data.

The operation determining section 21b is a circuit section or program software configured to in response to an input from the operation section 13, determine an operation instruction signal inputted. Upon receipt of a result of the determination made by the operation determining section 21b, the image processing/control section 21 performs control of the corresponding component unit.

The communication control section 21c is a circuit section or program software configured to control the communication section 12. In response to an instruction signal from, e.g., the operation section 13, the communication control section 21c performs control related to communication, for example, performs control of selection of, e.g., whether communication between terminal apparatuses or communication with the file server 30 via the network 40 is to be performed, and the selected communication means to perform various corresponding communication control.

The display control section 21d is a circuit section or program software configured to control the display section 15. In response to an instruction signal from, e.g., the operation section 13, the display control section 21d performs control related to display, for example, control for selection of or switching to the content to be displayed on the display panel of the display section 15.

The recording control section 21e is a circuit section or program software configured to control the recording section 16. In response to an instruction signal from, e.g., the operation section 13, the recording control section 21e performs control related to recording, for example, control to convert various information recorded in the recording section 16 into a predetermined data format and recording the resulting data in a predetermined area of a predetermined storage medium (not illustrated).

The image determining section 21f is a circuit section or program software configured to perform processing for determining various information obtained by image data analysis based on, e.g., image data acquired by the image pickup section 11 or image data transferred and inputted from, e.g., another terminal apparatus, for example, processing for determining, e.g., information on a light source in a shooting environment, information on a distance to an object or information on the object (for example, a face area).

The shooting information providing section 21g is a circuit section or program software configured to provide various corresponding information to, e.g., image data acquired using the image pickup section 11 or image data transferred and inputted from, e.g., another terminal apparatus. Various information provided by the shooting information providing section 21g includes, e.g., medical meta data, which has been described above, in addition to ordinary meta data (time/date information, apparatus information, etc.) acquired at the time of acquisition of image data (shooting).

Here, information included in medical meta data includes, in addition to various information described above with reference to FIG. 3, for example, information relating to communication (communication information), patient information (subject information), information relating to a doctor, information relating to the doctor's order (order information and request information), information relating to treatment or a test of a diseased part (condition information), information relating to an apparatus (e.g., apparatus identification information (e.g., a GS1 code, a JAN code and an apparatus serial number)), color chart information, measure information, information relating to a position and a location of an apparatus, information on approval as a medical image or a medical reference image and authenticity information, and various other information.

Next, a major configuration of the file server 30 in the medical support system 1 according to the present embodiment will be described. The file server 30 corresponds to, for example, a file server included in, e.g., an existing hospital information system (HIS).

As illustrated in FIG. 9, the file server 30 mainly includes, e.g., a control section 31, an image database 32, a database 33 for order information, etc., a patient information database 34 and a communication section 35.

The control section 31 is a control circuit configured to perform overall control of the file server 30. The control section 31 mainly includes, e.g., a patient determining section 31a, an order information creating section 31b and a shooting condition information creating section 31c.

The patient determining section 31a is a circuit section or program software for associating patient image data transferred from an information terminal apparatus (10, 20) with corresponding patient information. The patient determining section 31a performs patient determination based on, e.g., medical meta data accompanying patient image data, the image database 32 and the patient information database 34.

The order information creating section 31b is a circuit section or program software configured to when a doctor accesses the file server 30 to create order information, support creation of order information with reference to various information such as the image database 32, the database 33 for order information, etc., and the patient information database 34.

For example, a certain format of order information relating to image shooting is prescribed on a diseased part-by-diseased part basis. Therefore, at the time of creation of order information, a doctor can create a rough form of order information by selecting, e.g., a desired test type (for example, “image shooting test”) and a desired diseased part (for example, “face” or “knee”).

In addition, in the medical support system 1 according to the present embodiment, for example, electronic medical record information, test history information, etc., of the subject patient are referred to from the patient information database 34, and if an order that is the same as or similar to an order for a test or the like to be conducted this time (for example, “image shooting test”) is found in a list of orders in tests and the like conducted in the past, e.g., such order information can be read to create new order information.

As described above, the order information creating section 31b is a component section configured to when a doctor creates order information, support the order information creation by omitting input work or enhancing efficiency.

The shooting condition information creating section 31c is a circuit section or program software configured to when order information created by the order information creating section 31b based on an instruction from a doctor is, for example, an order involving image shooting, create shooting condition information suitable for the image shooting instruction.

Based on patient information, etc., referred to at the time of creation of order information, the shooting condition information creating section 31c creates shooting condition setting information optimum for image shooting corresponding to the order information of this time with reference to, e.g., past order information at the time of a similar “image shooting test” conducted in the past and information on a result of the test (acquired and recorded image data and accompanying information, etc.).

Normally, when a doctor creates order information, the doctor only provides an instruction relating to medical practice, but in the medical support system 1 according to the present embodiment, based on the order information created by the doctor, the order information including an instruction relating to medical practice only, the shooting condition information creating section 31c creates shooting condition information for image shooting. The shooting condition information is transmitted to an information terminal apparatus (10, 20) that receives the order information and shoots (acquires) image data, and is then used for shooting.

On the other hand, the image database 32 is a database section with image data accumulated. A plurality of image data accumulated in the image database 32 includes, e.g., sample images each prepared in advance to be used as a reference when an image of a particular diseased part or a specific symptom is acquired, in addition to various image data included in respective specific patient information pieces, for example, a medical image or medical reference image shot of a diseased part or a symptom of a particular patient and an image for identifying a patient, the image including a face.

Here, a specific example of a medical image or a medical reference image includes, e.g., image data which is recorded indicating a situation during a course of medical care, before or after surgery and image data which is recorded indicating a state of a diseased part during surgery. Also, examples of an image data form in the case include, e.g., X-ray image data, ultrasound image data and endoscopic image data in addition to image data recorded in a general image pickup apparatus. From among these data, e.g., X-ray image data, ultrasound image data, endoscopic image data are image data each acquired in response to a doctor's order (request) by a person having a healthcare-related qualification, using a dedicated apparatus installed in a medical base, the image data including medical images each approved by the relevant doctor.

The plurality of image data accumulated in the image database 32 other than the sample images are recorded in a form in which the plurality of image data associated with respective healthcare-related information pieces (electronic medical record information, patient information, etc.). Furthermore, in each image data, meta data including, e.g., shooting time/date information, and medical meta data further provided with various information based on a patient ID and electronic medical record information are recorded so as to accompanying the image data.

On the other hand, the database 33 for order information, etc., is a database section configured to accumulate information relating to order information provided by doctors. Examples of information recorded in the database 33 for order information, etc., include sample information of order specification forms each set in advance for a predetermined diseased part or a predetermined symptom. In each order specification form, for example, a doctor name (requester name), a patient ID, used apparatus information and shooting condition information are stated.

When a doctor requests each medical division to acquire image data in order to conduct a test or the like of a particular patient, it is necessary to issue order information according to a diseased part or a symptom of the patient on a subject patient-by-subject patient basis for the test or the like.

This case provides the advantage of eliminating a need for a doctor to create order information from scratch. In other words, a doctor can refer to an order specification form matching a diseased part or a symptom of a subject patient for which the doctor creates order information from among a plurality of order specification forms (samples) recorded in the database 33 for order information, etc., and thus can create optimum order specification forms according to symptoms, diseased parts, etc., of individual subject patients.

The patient information database 34 is a database section with patient information, for example, patient IDs and electronic medical record information accumulated.

The communication section 35 is a circuit section or program software configured to transmit/receive various data files to/from the plurality of information terminal apparatuses (10, 20) via the network 40. The communication section 35 is controlled so as to perform predetermined communication, by a communication control section (not illustrated) included in the control section 31.

Operation of the medical support system 1 according to the present embodiment configured as described will be described.

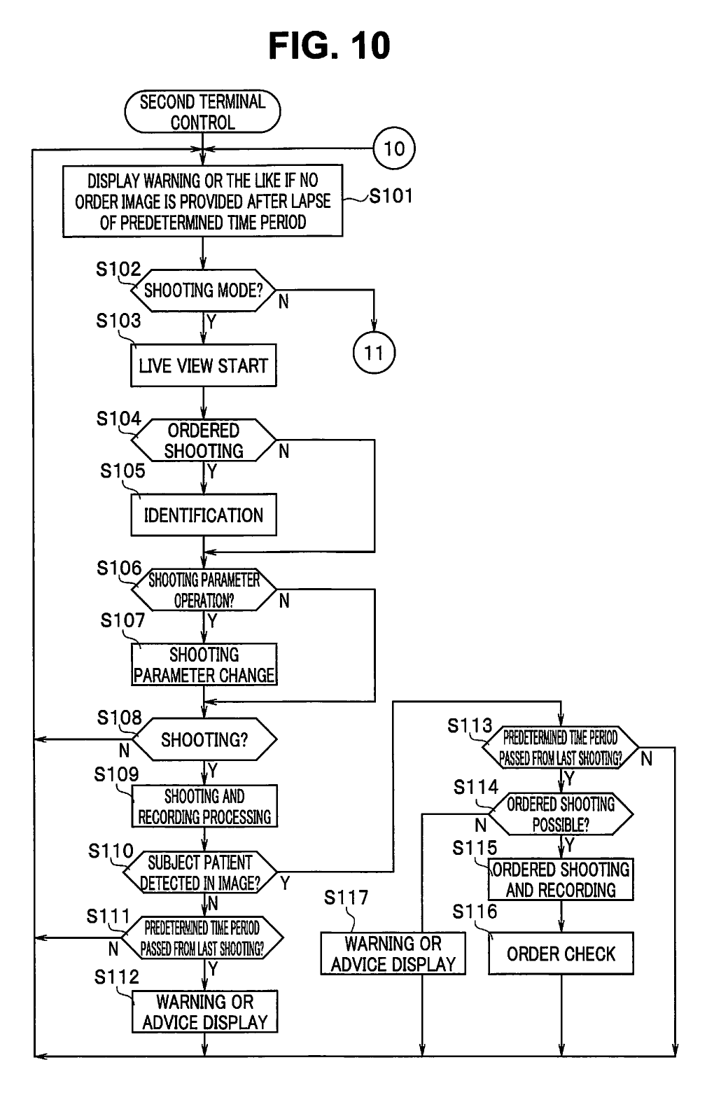

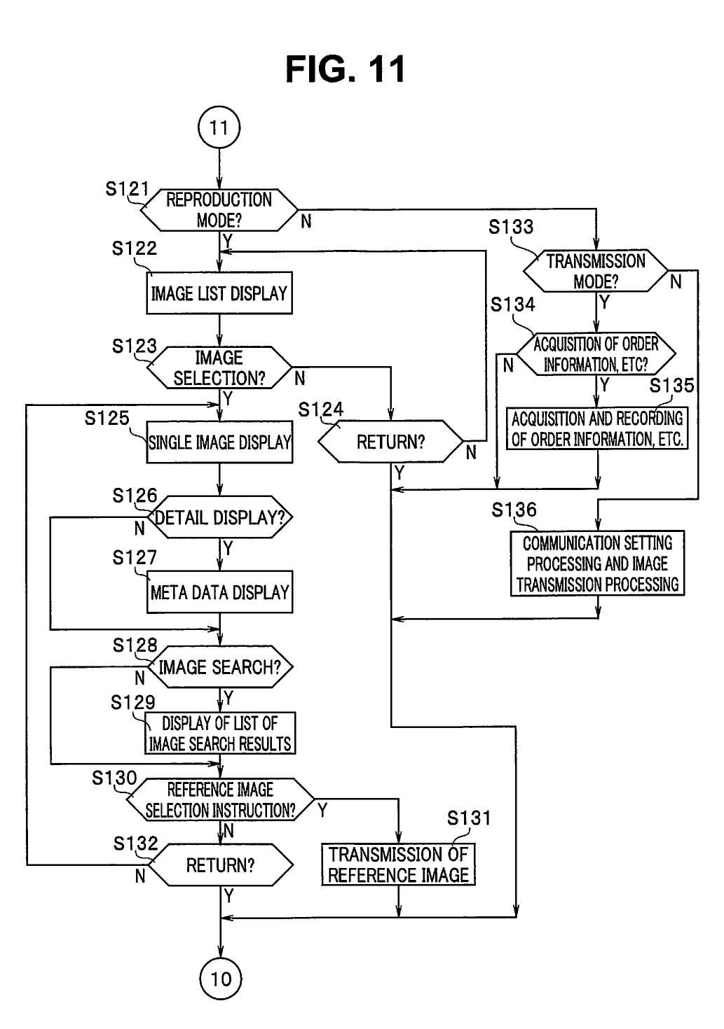

First, operation of a second terminal 20 from among the information terminal apparatuses in the medical support system 1 according to the present embodiment will be described. FIGS. 10 and 11 are flowcharts illustrating operation of a second terminal 20 from among the information terminal apparatuses in the medical support system according to the present embodiment.

In the medical support system 1 according to the present embodiment, as described above, a second terminal 20 is an information terminal apparatus to be used by, for example, a person other than a doctor.

First, it is assumed that the second terminal 20 is in a power-on state and thus has been activated to be usable.

In this state, first, if no image according to a doctor's order has been shot even after a lapse of a predetermined time period from a point of time of the activation of the second terminal 20, in step S101 in FIG. 10, the image processing/control section 21 in the second terminal 20 controls the display section 15 via the display control section 21d to provide predetermined warning display or the like. Here, examples of the predetermined warning display include warning display or the like for reminding shooting an ordered image of a subject patient. Subsequently, the operation proceeds to the processing in next step S102.

In step S102, the image processing/control section 21 determines whether or not the currently-set operation mode is a shooting mode. Here, if the shooting mode has been set, the operation proceeds to the next processing in step S103. Also, if an operation mode other than the shooting mode has been set, the operation proceeds to the processing in next step S121 in FIG. 11 (see circled reference numeral 11 in FIGS. 10 and 11).

In step S121 in FIG. 11, whether or not the currently-set operation mode is a reproduction mode is determined. Here, if the reproduction mode has been set, the operation proceeds to the processing in next step S122. Also, if an operation mode other than the reproduction mode has been set, the operation proceeds to the processing in step S133 (see circled reference numeral 11 in FIGS. 10 and 11).

In step S133 in FIG. 11, the image processing/control section 21 confirms whether or not the currently-set operation mode is a transmission mode. Here, if the transmission mode has been set, the operation proceeds to the processing in next step S136, and in step S136, the image processing/control section 21 performs, for example, communication setting processing and image transmission processing for transmitting a predetermined image.

An overview of the communication setting processing performed here is as follows. First, the image processing/control section 21 controls the display section 15 via the display control section 21d to display a predetermined setting menu screen. A user performs a desired setting operation while viewing the setting menu screen. In response to the setting operation, the operation determining section 21b in the image processing/control section 21 determines an inputted operation instruction signal and performs various setting processing according to the operation instruction signal.

Also, an image transmitted as a result of the image transmission processing performed here is a medical reference image (watching-over image) meeting a predetermined shooting condition from among images of the subject patient, the images being acquired by the second terminal 20.

Subsequently, the operation returns to the processing in step S101 in FIG. 10 and repeats the subsequent processing (see circled reference numeral 10 in FIGS. 11 and 10).

On the other hand, if it is determined in the processing in step S133 above that a mode other than the transmission mode has been set, the operation proceeds to the processing in step S134, and in step S134, the image processing/control section 21 determines whether or not order information or the like is received from the first terminal 10.

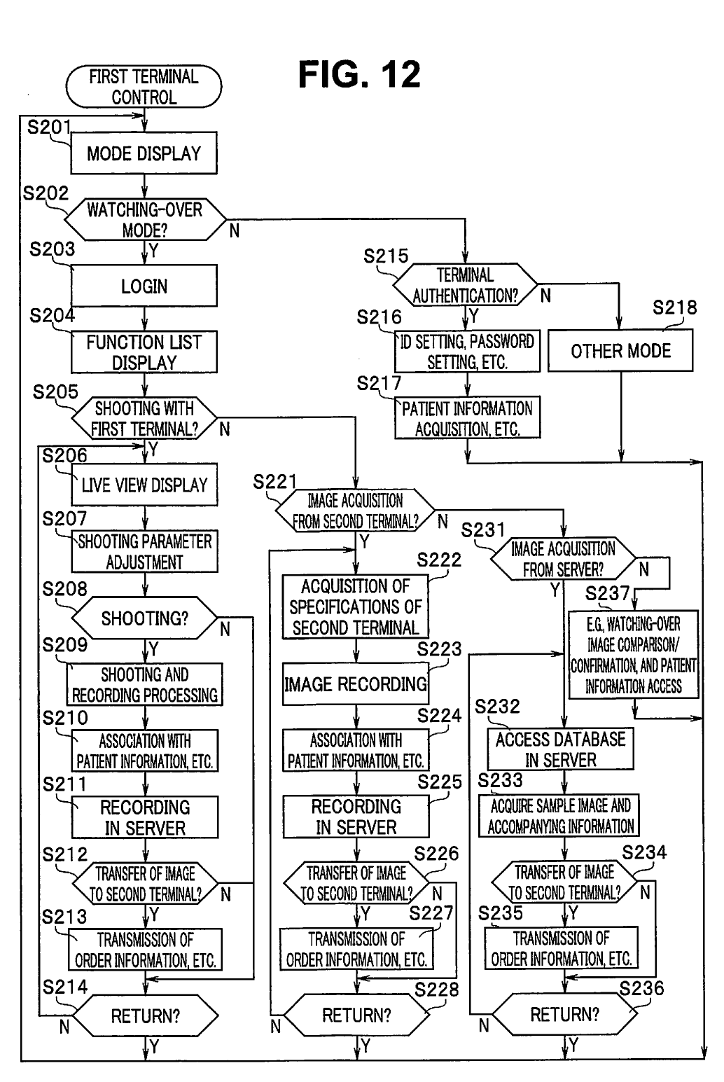

The determination processing performed here is processing performed in response to later-described order information transmission processing performed by the first terminal 10 (see the processing in steps S213, S227 and S235 in FIG. 12).

During the order information transmission processing being performed by the first terminal 10, if the second terminal 20 is in the transmission mode, the second terminal 20 can receive order information from the first terminal 10. Therefore, the user can receive the order information by performing a predetermined operation for receiving the order information.

Here, if it is determined that an instruction to receive order information or the like has been provided, the operation proceeds to the processing in next step S135. Also, if it is determined that an instruction not to receive order information or the like has been provided or no transmission according to order information transmission processing performed by the first terminal 10 has occurred, the operation returns to the processing in step S101 in FIG. 10 and repeats the subsequent processing (see circled reference numeral 10 in FIGS. 11 and 10).

In step S135, the image processing/control section 21 performs processing for receiving order information or the like and processing for recording the received information. Upon the second terminal 20 acquiring the order information or the like from the first terminal 10 as described above, the second terminal 20 can set a shooting operation mode (also referred to as “watching-over shooting mode”) based on the order information.

Subsequently, the operation returns to the processing in step S101 in FIG. 10 and repeats the subsequent processing (see circled reference numeral 10 in FIGS. 11 and 10).

On the other hand, if it is determined in the processing in step S102 above that the shooting mode has been set and the operation proceeds to the processing in step S103, in step S103, the image processing/control section 21 controls the image pickup section 11 via the image pickup control section 21a and controls the display section 15 via the display control section 21d to start operation for displaying a predetermined live view image.

Subsequently, in step S104, the image processing/control section 21 determines whether or not to ordered shooting based on the doctor's order (e.g., a request or an instruction) (based on the order information) is performed. This determination is, for example, determination of a signal of a predetermined instruction (predetermined shooting mode selection operation) provided by the user or determination of whether or not order information has been transferred to the second terminal 20 in advance.

Here, if it is determined that an ordered shooting instruction has been provided, the operation proceeds to the processing in next step S105. Also, it is determined that no ordered shooting instruction has been provided, the processing proceeds to step S106.

In step S105, the image processing/control section 21 performs identification processing. The identification processing performed here may be, for example, determination of whether or not the relevant person is the subject patient registered in advance by means of face detection based on the live view image or confirmation based on a voice of the subject patient in response to asking the subject patient. For example, voice recording is performed when a health professional on the testing side asks, e.g., “Are you xxx (patient name)?” and in response to the asking, the patient replies “Yes”. Then, e.g., a method in which a voice print is authenticated based on the voice data is contemplated.

Next, in step S106, the image processing/control section 21 monitors an instruction signal from the operation determining section 21b to determine whether or not a shooting parameter operation has been performed. Here, if a shooting parameter operation has been performed, the operation proceeds to the processing in next step S107. Also, if no shooting parameter operation has been performed, the operation proceeds to the processing in step S108.

Subsequently, in step S107, the image processing/control section 21 performs shooting parameter change processing according to the shooting parameter operation instruction determined in the processing in step S106 above. Subsequently, the operation proceeds to the processing in step S108.

In step S108, the image processing/control section 21 determines whether or not an instruction signal to provide an instruction to perform a shooting operation has been generated. Here, if it is determined that such shooting instruction has been generated, the operation proceeds to the processing in next step S109. Also, if it is determined that no such shooting instruction has been generated, the operation returns to the processing in step S101 above and repeats the subsequent processing.

In step S109, the image processing/control section 21 controls the image pickup section 11 via the image pickup control section 21a to perform predetermined shooting processing and controls the recording section 16 via the recording control section 21e to perform predetermined recording processing. Examples of the shooting processing performed here include not only still image shooting operation but also various types of image shooting such as voice-included still image shooting and movie shooting (whether voice included or not).

In step S110, the image processing/control section 21 performs processing for detecting a particular subject (subject patient) in the image acquired as a result of the processing in step S109 above. Here, if the subject patient is detected, the operation proceeds to the processing in next step S113. Also, if the subject patient is not detected, the operation proceeds to the processing in step S111.

The object (subject patient) detection processing performed here may be performed based on the image data acquired in step S108 above or may be performed based on the running live view image.

In step S111, the image processing/control section 21 determines whether or not a predetermined time period has passed from last shooting (last activation). Here, if a predetermined time period has passed from last shooting (activation), the operation proceeds to the processing in next step S112. Also, if a predetermined time period has not passed from last shooting (activation), the operation returns to the processing in step S101 above and repeats the subsequent processing.

In next step S112, the image processing/control section 21 controls the display section 15 via the display control section 21d to provide predetermined warning display or advice display or the like. Subsequently, the operation returns to the processing in step S101 above and repeats the subsequent processing.

On the other hand, if the subject patient has been detected as a result of the processing in step S110 and the operation proceeds to processing in next step S113, in step S113, the image processing/control section 21 determines whether or not a predetermined time period has passed from last subject patient image shooting. Here, if a predetermined time period has not passed from last subject patient image shooting, the operation returns to the processing in step S101 above and repeats the subsequent processing.

On the other hand, in step S113, if a predetermined time period has passed from last shooting, the operation proceeds to the processing in next step S114 and in step S114, the image processing/control section 21 determines whether or not predetermined ordered shooting is possible. Here, processing for determining whether or not ordered shooting is possible is determination of whether or not shooting based on shooting conditions according to the order information received from the first terminal 10 and recorded in advance in step S135 in FIG. 11 above is possible under the current state of the second terminal 20.

This is processing for reflecting, e.g., an intention of the doctor that has issued the order. As a matter of course, the shooting conditions may include not only conditions relating to quality of an image obtained by shooting, but also conditions based on order information including “medical examination management information”.

If the branching processing in step S114 is processing based on such ordered conditions, determination of whether or not the ordered conditions are met may be performed by quantifying shooting results for various conditions and comparing the quantified shooting results with predetermined determination criteria. Examples of various conditions include a condition that a shooting subject person or region is different, a condition that contemplated equipment or accessory is not used, a condition that a designated composition is not performed, a condition that no designated shooting parameter can be set, a condition that an amount of exposure is insufficient, a condition that proper focusing cannot be performed and a condition that camera shake occurs. Also, it is possible to determine that the ordered conditions are not met, e.g., where a shooting sequence is not a prescribed sequence or where the number of shot images is in excess or short of a designated number of shot images.

Distinctive characteristics of medical examination include, e.g., determination of whether or not the ordered shooting is possible based on, e.g., information including information on whether or not patient identification has been performed based on the voice information or other intention indication input in the identification processing in the processing in step S105 and a result of the identification, and information on whether or not, e.g., shooter information is included. Such characteristics may be set as default on the system side or may be designated by, e.g., an attending doctor.

Here, if it is determined that the predetermined order shooting is possible, the operation proceeds to the processing in next step S115, and in step S115, the image processing/control section 21 performs predetermined watching-over shooting processing based on the predetermined order information and subsequently performs processing for recording the acquired image information (image data). Subsequently, the operation proceeds to the processing in step S116.

In step S116, the image processing/control section 21 performs order check processing for determining whether or not the image has been acquired according to the order information. Subsequently, the operation returns to the processing in step S101 above and repeats the subsequent processing.

The check processing here also may be performed under conditions similar to the conditions in step S114 above. Also, e.g., the check processing may be accompanied by warning or guide display that is similar to warning or guide display in step S117. For example, before shooting, a shooter may be worried about hand shake or try to concentrate on a composition, and thus an image often changes. Therefore, in some cases, whether or not the conditions are met cannot completely be determined. Therefore, it is important to check after shooting whether or not the conditions have been met as described above.

Furthermore, at this timing, it is also possible to transmit, e.g., the image or characteristics of the image and as necessary, voice information to an external device (e.g., the system in the hospital) to request determination of whether or not the conditions are met.

In this case, accurate person recognition and region determination can be performed at high speed and high precision by, e.g., a large computer and thus, determination, advice and guide can be performed for a large amount of information.

Also, as necessary, e.g., deep learning may be performed based on these pieces of information and a result of the deep learning may be used concurrently for the determination. For example, for, e.g., a particular case, whether or not the relevant image really will do, that is, whether or not the shooting is correct, can be determined by deep learning using a plurality of images of cases that is the same as the particular case. Determination of, e.g., a face image for identifying a patient cannot easily be performed depending on the disease state, but use of artificial intelligence is highly likely to enable the determination.

In consideration of a result of the determination, at this timing, reflection of a result of the recording performed in the processing in step S115 may be performed or the recording may be performed again. Here, it is important to include information enabling distinguishing between normal shooting and shooting based on an order, and, e.g., a result of check based on order conditions may be recorded in association with the image.

In this case, a particular file format is employed to include image information and such order-related, image management information (e.g., the content of the order and/or a result of the determination of whether or not the order is met or only a part of such pieces of information) according to particular rules.

In other words, it is possible that: shooting condition information created based on order information is received from the file server; image data is acquired by performing image shooting based on the shooting condition information; an image file having a format in which the acquired image data and the shooting condition information are associated with each other; and the image file is recorded in the recording section.

On the other hand, if it is determined in the processing in step S114 above that ordered shooting is impossible, the operation proceeds to the processing in step S117, and in step S117, the image processing/control section 21 performs predetermined warning or advice display. Subsequently, the operation returns to the processing in step S101 above and repeats the subsequent processing.

Here, since ordered shooting needs to be shooting meeting various elements that are important in medical examination, re-shooting may be suggested by, e.g., warning or shooting advice or shooting guide that is easier to understand may be indicated. Such suggestion, advice, guide, etc., may be indicated by, for example, an image or voice or by both of an image and voice.

If the order conditions cannot be met even under the shooting conditions based on the order information, guide information (including, e.g., a warning or an advice) is provided to easily obtain an image that can be readily used in a medical institution.

Also, as described above, if it is determined in the processing in step S121 in FIG. 11 that the reproduction mode has been set and the operation proceeds to the processing in step S122 in FIG. 11, in step S122, the image processing/control section 21 controls the display section 15 via the display control section 21d to perform predetermined image list display processing using the display panel (not illustrated) of the relevant display section 15. The image list display processing is, for example, processing for displaying a list of images based on a plurality of image data recorded in the storage medium (not illustrated) included in the recording section 16 of the second terminal 20. The image list display processing is similar to processing performed by an information terminal apparatus having a general form (e.g., a camera, a smartphone or a tablet PC).

Subsequently, in step S123, the image processing/control section 21 determines whether or not a selection instruction signal for selecting a predetermined image has been generated, via the operation determining section 21b. Here, if it is determined that the image selection instruction signal has been generated, the operation proceeds to next step S125. Also, if it is determined that no image selection instruction signal has been generated, the operation proceeds to the processing in step S124, and in step S124, whether or not an instruction signal for coming out of the reproduction mode and returning to the previous operation mode has been generated is determined.

Here, if it is determined that such instruction signal for returning has been generated, the operation returns to the processing in step S101 in FIG. 10 and repeats the subsequent processing (see circled reference numeral 10 in FIGS. 11 and 10). Also, if it is determined that no such instruction signal for returning has been generated, the operation returns to the processing in step S122 and repeats the subsequent processing. In other words, the image list display is continued.

In step S125, the image processing/control section 21 controls the display section 15 via the display control section 21d to perform single image display (enlarged display) processing for an image corresponding to the image selection instruction signal generated in the processing in step S123 above. Subsequently, the operation proceeds to the processing in step S126.

In step S126, the image processing/control section 21 determines whether or not a detail display instruction signal has been generated, via the operation determining section 21b. Here, if it is determined that a detail display instruction signal has been generated, the operation proceeds to the processing in next step S127. Also, if it is determined that no detail display instruction signal has been generated, the operation proceeds to the processing in step S128.

In step S127, the image processing/control section 21 controls the recording section 16 via the recording control section 21e to read meta data accompanying image data that is currently displayed on the display section 15. Subsequently, the image processing/control section 21 controls the display section 15 via the display control section 21d to display details of the meta data. Subsequently, the operation proceeds to the processing in step S128.

The meta data detail display automatically returns to the previous single image display after a lapse of a predetermined time period after the meta data detail display being provided. Also, alternatively, the meta data detail display may be terminated in response to a predetermined operation performed by a user and may return to the previous single image display. Furthermore, as another form, a display form of the meta data detail display may be made to be a form in which the meta data detail display is displayed so as to be superimposed on the single image display.

In step S128, the image processing/control section 21 determines whether or not predetermined image search processing is performed for the plurality of image data recorded on the storage medium (not illustrated) included in the recording section 16 of the second terminal 20. The image search processing contemplated in this case is, for example, a search for images including the face of the subject patient.

Here, if it is determined that an instruction to perform the image search processing has been provided, the operation proceeds to the processing in next step S129. Also, if it is determined that an instruction to perform the image search processing has been provided, the operation proceeds to the processing in step S130.

In step S129, after performing the image search processing, the image processing/control section 21 controls the display section 15 via the display control section 21d to display a list of results of the search.

The search result list display in this case corresponds to, for example, an operation of the patient's family member 120 when the patient's family member 120 shows patient images in the second terminal 20 to the doctor in the first pattern of use of the medical support system 1 described with reference to FIG. 2.

In step S130, the image processing/control section 21 determines whether or not a selection instruction signal to select a predetermined image as a reference image has been generated, via the operation determining section 21b.

An image selection operation in this case corresponds to an operation of the doctor 100 when the doctor 100 selects a candidate image for a medical reference image from among the patient images in the second terminal 20 in the first pattern of use of the medical support system 1 described with reference to FIG. 2.

Here, if it is determined that the reference image candidate selection instruction signal has been generated, the operation proceeds to the processing in step S131, and in step S131, processing for transmitting the selected reference image candidate to the first terminal 10 is performed.

The processing for transmission of the reference image candidate from the second terminal 20 to the first terminal 10 performed here corresponds to the later-described processing in step S221 in FIG. 12.

Also, if no reference image candidate selection instruction signal has been generated, the operation proceeds to step S132, and in step S132, the image processing/control section 21 determines whether or not a return instruction signal has been generated. Here, if it is determined that a return instruction signal has been generated, the operation returns to the processing in step S101 in FIG. 10 and repeats the subsequent processing (see circled reference numeral 10 in FIGS. 11 and 10). Also, if it is determined that no return instruction signal has been generated, the operation proceeds to the processing in step S125 and repeats the subsequent processing.

Next, operation of a first terminal 10 in the medical support system 1 according to the present embodiment will be described. FIG. 12 is a flowchart illustrating operation of an information terminal apparatus (first terminal 10) used by a doctor from among information terminal apparatuses in the medical support system according to the present embodiment.

In step S201 in FIG. 12, the image processing/control section 21 controls the display section 15 via the display control section 21d to display a currently-set operation mode. Subsequently, the operation proceeds to the processing in step S202.

Next, in step S202, the image processing/control section 21 determines whether or not the currently-set operation mode is the watching-over shooting mode from among the shooting modes. In this case, as described above, the watching-over shooting mode refers to a shooting operation mode based on order information from a doctor.

Here, if the watching-over shooting mode is currently set, the operation proceeds to the processing in next step S203. Also, if the watching-over shooting mode is not currently set, the operation proceeds to the processing in step S215.

In step S215, the image processing/control section 21 determines whether or not terminal authentication processing is performed. Here, the terminal authentication processing refers to a procedure for registering the first terminal 10 in the medical support system 1 in order to make the first terminal 10 usable in the medical support system 1. The terminal authentication processing needs to be performed just once when the information terminal apparatus is used in the medical support system 1 for the first time. From the second time onwards, by using of an ID and a password set and acquired by the terminal authentication processing the authenticated first terminal 10 can be used in the medical support system 1.

Here, if the terminal authentication processing is performed, the operation proceeds to the processing in next step S216. Also, if no terminal authentication processing is performed, the operation proceeds to the processing in step S218, and in step S218, the image processing/control section 21 performs processing in another operation mode. Here, examples of the other operation mode include, e.g., a telephone communication mode and an e-mail transmission/reception mode. However, these other operation modes are not directly related to the present invention, and detailed description and illustration of such operation modes will be omitted.

As described above, when the terminal authentication processing is performed, the operation proceeds to the processing in step S216, and in the step S216, the image processing/control section 21 performs processing relating to a procedure necessary for terminal authentication such as ID setting processing and password setting processing is performed. Subsequently, the operation proceeds to the processing in step S217.

In step S217, the image processing/control section 21 performs, e.g., processing for acquiring patient information, etc., of a subject patient from the file server according to an operation instruction signal inputted via the operation determining section 21b. Upon end of the necessary processing, the operation returns to the above-described processing in step S201 in response to an end instruction signal and repeats the subsequent processing.

In the above-described processing in step S202, if the watching-over shooting mode is currently set, the operation proceeds to the processing in next step S203 and in step S203, the image processing/control section 21 performs login processing using the ID and the password acquired in the above-described processing in step S215 (terminal authentication processing).

Subsequently, in step S204, the image processing/control section 21 controls the display section 15 via the display control section 21d to perform function list display.

Next, in step S205, the image processing/control section 21 determines whether or not shooting is performed using the first terminal 10. In this case, shooting using the first terminal 10 corresponds to, for example, an operation when the doctor 100 shoots a patient image using the first terminal 10 and sets the patient image as a sample image for a medical reference image in the second pattern of use in the medical support system 1 described with reference to FIG. 4. In other words, a shooting operation (image pickup operation) performed here is means for acquiring a sample image for a medical reference image of the subject patient.

Here, if shooting is performed using the first terminal 10, the doctor performs, for example, an operation to select and designate an application for performing a shooting operation from the function list displayed on the display section 15. As described above, if it is determined that shooting is performed using the first terminal 10, the operation proceeds to the processing in next step S206. Also, if no image pickup operation is performed using the first terminal 10, the operation proceeds to the processing in step S221.

In step S206, image processing/control section 21 controls the image pickup section 11 via the image pickup control section 21a and controls the display section 15 via the display control section 21d to start an operation of displaying a predetermined live view image.

Next, in step S207, the image processing/control section 21 monitors an instruction signal from the operation determining section 21b to determine whether or not a shooting parameter operation has been performed, and if it is determined that a shooting parameter operation has been performed, the image processing/control section 21 performs corresponding shooting parameter adjustment processing according to an instruction of the shooting parameter operation. Subsequently, the operation proceeds to the processing in step S208.

In step S208, the image processing/control section 21 determines whether or not an instruction signal to provide an instruction for performing an image pickup operation has been generated. Here, if it is determined that the shooting instruction signal has been generated, the operation proceeds to the processing in next step S209. Also, if it is determined that no shooting instruction signal has been generated, the operation proceeds to the processing in step S214.

In step S209, the image processing/control section 21 controls the image pickup section 11 via the image pickup control section 21a to perform predetermined image pickup processing and controls the recording section 16 via the recording control section 21e to perform predetermined recording processing.

Subsequently, in step S210, the image processing/control section 21 associates the patient information, etc., acquired from the file server 30 (see step S217) with the image data acquired in step S209 above.

Next, in step S211, the image processing/control section 21 performs processing for recording the image data in a predetermined area (image database 32) in the file server 30. This processing includes processing for transferring the image data to the file server 30 via the network 40.

Next, in step S212, the image processing/control section 21 determines whether or not the image data is transferred to the second terminal 20.

Here, if the image data is transferred to the second terminal 20, the doctor performs, for example, an operation to select and designate the application for performing an operation of image transfer to/from another information terminal apparatus from the function list displayed on the display section 15.

If it is determined that image transfer from the first terminal 10 to the second terminal 20 is performed, the operation proceeds to the processing in next step S213. Also, if it is determined that no image transfer from the first terminal 10 to the second terminal 20 is performed, the operation proceeds to the processing in step S214.

In step S213, the image processing/control section 21 transmits order information, etc., to the second terminal 20.

The order information transmission processing performed here is processing performed in the second terminal 20 in response to order information acquisition processing (see the processing in step S134 in FIG. 11).

If the second terminal 20 is in a transmission mode during the order information transmission processing being performed in the first terminal 10, the second terminal 20 can receive the order information from the first terminal 10.

Next, in step S214, the image processing/control section 21 determines whether or not a return instruction signal has been generated. Here, if it is determined that a return instruction signal has been generated, the operation returns to the above-described processing in step S201 and repeats the subsequent processing. Also, if it is determined that no return instruction signal has been generated, the operation returns to the above-described processing in step S206 and repeats the subsequent processing.

On the other hand, if no shooting is performed using the first terminal 10 in the processing in the above-described processing in step S205, the operation proceeds to the processing in step S221, and in step S221, the image processing/control section 21 determines whether or not an image is acquired from the second terminal 20.

In this case, acquisition of an image from the second terminal 20 corresponds to, for example, an operation when the doctor 100 acquires a candidate image for a medical reference image from among the patient images in the second terminal 20 in an operation in the first pattern of use of the medical support system 1 described with reference to FIGS. 2 and 3. In other words, the image acquisition operation performed here is another means for acquiring a sample image for a medical reference image of the subject patient.

As described above, the second terminal 20 is, for example, an information terminal apparatus privately owned and used by a patient's family member or the like. Therefore, the second terminal 20 is not subjected to terminal authentication in the medical support system 1 and thus cannot access the file server 30. Therefore, here, an example based on an assumption that: the image data in the second terminal 20 is once transferred to the first terminal 10 owned by the doctor; and upon receipt of the image data, the doctor provides predetermined medical meta data to the image data via the first terminal 10 to approve the image data as a medical reference image and then transfers the image data to the file server 30 using the first terminal 10 formally authenticated is indicated.

Here, if an image is acquired from the second terminal 20, the doctor performs, for example, an operation to select and designate the application for performing an operation of image transfer to/from another information terminal apparatus in the function list displayed on the display section 15. Then, if it is determined that an operation of image acquisition from the second terminal 20 is performed, the operation proceeds to the processing in next step S222. Also, if it is determined that no operation of image acquisition from the second terminal 20 is performed, the operation proceeds to the processing in step S231.

In step S222, the image processing/control section 21 acquires, e.g., the predetermined image data and specification information of the second terminal 20 from the second terminal 20 (receives the information transmitted in the processing in step S131 in FIG. 11).

Here, the specification information of the second terminal 20 is acquired for the following reasons.

For example, it is assumed that an instruction to perform macro shooting is included in order information when shooting condition information is created according to the order information. In this case, a second terminal 20 to be used may have no macro mode for performing macro shooting as specifications of the second terminal 20. In order to respond to such case, the specifications of the second terminal 20 are grasped.

In other words, grasping the specifications of the second terminal 20 in advance enables creating shooting condition information according to the specifications of the second terminal 20 when shooting condition information is created according to order information.

More specifically, for example, in the case of a second terminal 20 having no macro mode, the second terminal 20 can consequently acquire an image with a desired shooting magnification using electronic image processing as a substitute of, e.g., an optical zoom function or an electronic zoom function. As described above, where a shooting instruction that is not set in the terminal specifications is provided, setting information that serves as a substitute for such shooting instruction can be included in shooting condition information.

Subsequently, in step S223, the image processing/control section 21 receives image data transferred from the second terminal 20 and controls the recording section 16 via the recording control section 21e to perform predetermined recording processing.

Next, in step S224, the image processing/control section 21 associates the patient information, etc., acquired from the file server 30 (see step S217) with the image data acquired at step S223 above.

Next, in step S225, the image processing/control section 21 performs processing for recording the image data to the predetermined area (image database 32) in the file server 30. This processing includes processing for transferring the image data to the file server 30 via the network 40.

Next, in step S226, the image processing/control section 21 determines whether or not the image data is transferred to the second terminal 20. In this case, the image data to be transferred to the second terminal 20 is image data approved as a medical reference image by the doctor by the patient information, etc., being added as meta data to the image data.

Here, if the image data is transferred to the second terminal 20, the doctor performs, for example, an operation to select and designate the application for performing an operation of image transfer to/from another information terminal apparatus in the function list displayed on the display section 15.

If it is determined that the image transfer from the first terminal 10 to the second terminal 20 is performed, the operation proceeds to the processing in next step S227. Also, if no image transfer from the first terminal 10 to the second terminal 20 is performed, the operation proceeds to the processing in step S228.

In step S227, the image processing/control section 21 transmits the order information, etc., to the second terminal 20.

The order information transmission processing performed here is processing performed in response to the above-described order information acquisition processing in the second terminal 20 (see the processing in step S134 in FIG. 11).

If the second terminal 20 is in a transmission mode during the order information transmission processing being performed in the first terminal 10, the second terminal 20 can receive the order information from the first terminal 10.

Next, in step S228, the image processing/control section 21 determines whether or not a return instruction signal has been generated. Here, if it is determined that a return instruction signal has been generated, the operation returns to the above-described processing in step S201 and repeats the subsequent processing. Also, if it is determined that no return instruction signal has been generated, the operation returns to the above-described processing in step S222 and repeats the subsequent processing.

If it is determined in the above-described processing in step S221 that no image acquisition from the second terminal 20 is performed, the operation proceeds to the processing in step S231, and in step S231, the image processing/control section 21 determines whether or not an image is acquired from the file server 30. In this case, acquisition of an image from the file server 30 corresponds to, for example, an operation when the doctor 100 accesses the file server 30 and acquires a desired sample image using the first terminal 10 in the third pattern of use of the medical support system 1 described with reference to FIG. 5. In other words, the image acquisition operation performed here is another means for acquiring a sample image for a medical reference image of the subject patient.

Here, if an image is acquired from the file server 30, the doctor performs, for example, an operation to select and designate the application for performing an operation of image transfer to/from the file server in the function list displayed on the display section 15. Consequently, if it is determined that an operation of image acquisition from the file server 30 is performed, the operation proceeds to the processing in next step S232. Also, if no operation of image acquisition from the file server 30 is performed, the operation proceeds to the processing in step S237, and in step S237, the image processing/control section 21 performs other processing, for example, medical reference image (watching-over image) comparison/confirmation processing or patient information access processing. Subsequently, the operation returns to the above-described processing in step S201 and repeats the subsequent processing.

On the other hand, if it is determined in the processing in step S231 above that an image is acquired from the file server 30, the operation proceeds to next step S232, and in step S232, the image processing/control section 21 communicates with the file server 30 via the network 40 and accesses, e.g., the image database 32, the database 33 for order information, etc., and the patient information database 34 to search for desired information. Here, the desired information to be searched for is sample image data according to, e.g., a symptom, a diseased status and/or a diseased part of the subject patient and various accompanying information.

In step S233, the image processing/control section 21 acquires the desired information searched for in the above-described processing in step S232 (e.g., the sample image data and the accompanying information).

Next, in step S234, the image processing/control section 21 determines whether or not the information acquired in step S233 above (e.g., the sample image and the accompanying information) is transferred to the second terminal 20.

Here, if the acquired information is transferred to the second terminal 20, the doctor performs, for example, an operation to select and designate the application for performing an operation of image transfer to/from another information terminal apparatus in the function list displayed on the display section 15.

If it is determined that the information transfer from the first terminal 10 to the second terminal 20 is performed, the operation proceeds to the processing in next step S235. Also, if no information transfer from the first terminal 10 to the second terminal 20 is performed, the operation proceeds to the processing in step S236.

In step S235, the image processing/control section 21 transmits the order information, etc., to the second terminal 20.

The order information transmission processing performed here is processing performed in response to the order information acquisition processing in the second terminal 20 (see the processing in step S134 in FIG. 11).

If the second terminal 20 is in the transmission mode during the order information transmission processing being performed in the first terminal 10, the second terminal 20 can receive the order information from the first terminal 10.

Next, in step S236, the image processing/control section 21 performs whether or not a return instruction signal has been generated. Here, if it is determined that return instruction signal has been generated, the operation returns to the processing in step S201 above and repeats the subsequent processing. Also, if it is determined that no return instruction signal has been generated, the operation returns to the above-described processing in step S232 and repeats the subsequent processing.

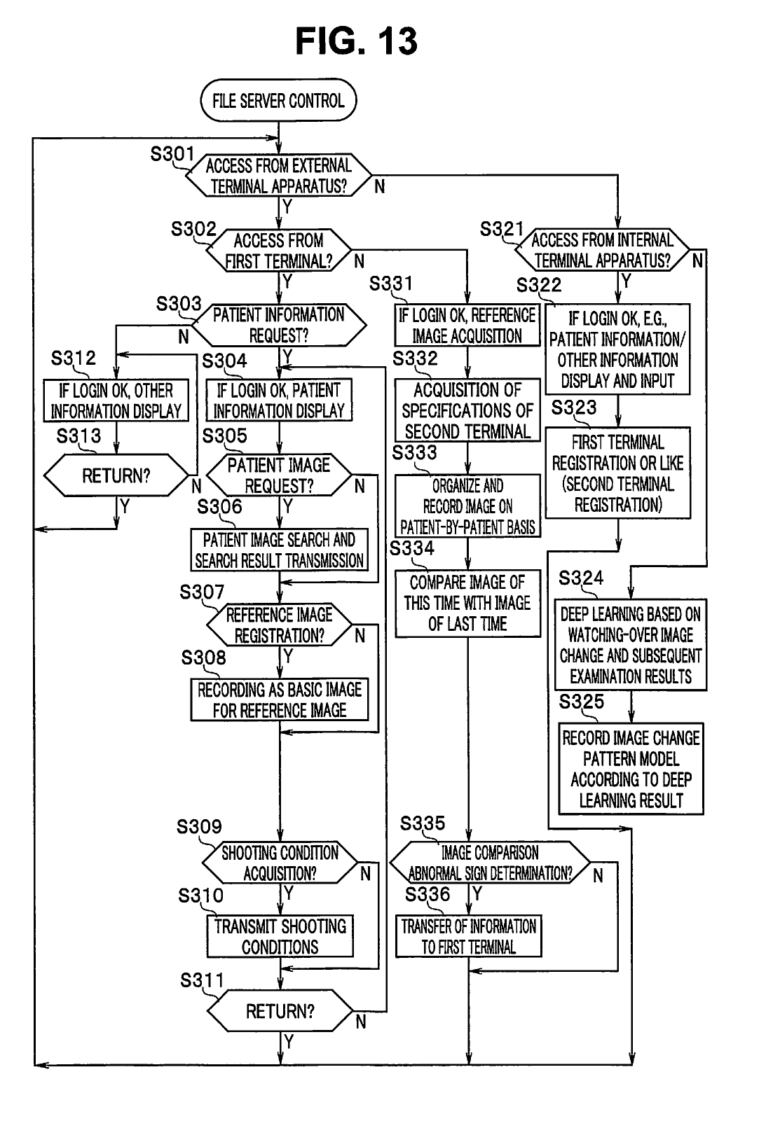

Next, operation of the file server 30 in the medical support system 1 according to the present embodiment will be described. FIG. 13 is a flowchart illustrating operation of the file server in the medical support system according to the present embodiment.

First, it is assumed that the file server 30 is in an activated state and also in a standby state in which the file server 30 waits for access from another device. In this state, in step S301 in FIG. 13, the control section 31 determines whether or not an external information terminal apparatus (10, 20) has accessed the file server 30 via the communication section 35. Here, if it is determined that an external information terminal apparatus (10, 20) has accessed the file server 30, the operation proceeds to the processing in next step S302. Also, if it is determined that a terminal apparatus other than the external information terminal apparatus (10, 20) has accessed the file server 30, the operation proceeds to the processing in step S321.

In step S302, the control section 31 determine whether or not the access via the communication section 35 is access from a first terminal 10. Here, if it is determined that the access is access from the first terminal 10, the operation proceeds to the processing in next step S303. Also, if it is determined that the access is access other than a terminal other than the first terminal 10 (that is, a second terminal 20), the operation proceeds to the processing in step S331.

In step S303, the control section 31 monitors an instruction signal from the first terminal 10, via the communication section 35 to determine whether or not patient information has been requested. Here, if patient information is requested from the first terminal 10, the operation proceeds to the processing in next step S304. Also, if an instruction signal from the first terminal 10 is not a patient information request, the operation proceeds to the processing in step S312.

In step S312, the control section 31 performs predetermined login procedure processing. In response to the processing, the first terminal 10 performs predetermined login procedure processing. Consequently, upon the login to the file server 30 being completed (if the login OK), e.g., processing for other information display is performed.

Although illustration is omitted in FIG. 13, here, if the first terminal 10 cannot login to the file server 30, the file server 30 cancels the subsequent processing and returns to the above-described processing in step S301 and enters a standby state.

Subsequently, in step S313, the control section 31 monitors an instruction signal from the first terminal 10, via the communication section 35 to determine whether or not a return instruction signal has been generated. Here, if it is determined that a return instruction signal has been generated, the operation returns to the above-described processing in step S301 and repeats the subsequent processing. Also, if it is determined that no return instruction signal has been generated, the operation returns to the above-described processing in step S312 and repeats the subsequent processing.

If it is determined in the processing in step S303 that the request from the first terminal 10 is a request for patient information and the operation proceeds to the processing in step S304, in step S304, the control section 31 performs predetermined login procedure processing (processing similar to the above-described processing in step S312). In response to the processing, the first terminal 10 performs predetermined login procedure processing. Consequently, upon the login to the file server 30 being completed (if the login OK), the control section 31 transfers the patient information requested in the above-described processing in step S303 to the first terminal 10. Upon receipt of the patient information, the first terminal 10 displays the patient information on the display section 15.

Although illustration is omitted also here, if the first terminal 10 cannot login to the file server 30, the file server 30 cancels the subsequent processing and returns to the above-described processing in step S301 and enters a standby state, which is similar to the above-described processing in step S312.

Subsequently, in step S305, the control section 31 determines whether or not a request for image data of the subject patient (referred to as “patient image”; more specifically, for example, image data including information on a feature of the face of the patient) has been received from the first terminal 10, via the communication section 35. In this case, patient image data requested by the first terminal 10 is used as information for identifying the patient. The first terminal 10 transfers, e.g., the patient face feature information acquired in the later-described processing in step S306 in addition to the patient information acquired in the above-described processing in step S303, to the second terminal 20 as described below. Consequently, these pieces of information serve as guide information for the second terminal 20 that has received the pieces of information to shoot a medical reference image.

Here, if a patient image request has been received from the first terminal 10, the operation proceeds to the processing in next step S306. Also, if no patient image request has been received from the first terminal 10, the operation proceeds to the processing in step S307.

Subsequently, in step S306, the control section 31 searches the image database 32 and the patient information database 34 for a requested patient image of the subject patient and transmits patient image data based on a result of the search to the first terminal 10. Subsequently, the operation proceeds to the processing in step S307.

In step S307, the control section 31 monitors an instruction signal from the first terminal 10, via the communication section 35 to determine whether or not a medical reference image (watching-over image) registration operation signal has been received. The medical reference image (watching-over image) registration operation performed here corresponds to an operation when the doctor operates the first terminal 10 to transmit a medical reference image of the subject patient to the file server 30 to record the medical reference image in the file server 30.

Here, if it is determined that a medical reference image (watching-over image) registration operation signal has been received, the operation proceeds to the processing in next step S308. Also, if it is determined that no such registration operation signal has been received, the operation proceeds to the processing in step S309.

In step S308, the control section 31 associates the patient image data received from the first terminal 10 with a corresponding patient information file in the patient information database 34 based on medical meta data accompanying the image data, and then records the image data in the image database 32.

Next, in step S309, the control section 31 monitors an instruction signal from the first terminal 10, via the communication section 35 to determine whether or not a shooting condition information acquisition operation instruction signal has been received.

In this case, the shooting condition information is information included in the patient information of the subject patient (displayed patient information in step S304), the information relating to shooting conditions for acquiring a series of image data according to, e.g., a symptom, a disease status and/or a diseased part of the patient (for example, a shooting angle, a shooting area and/or shooting apparatus setting information (various setting information such as exposure, focal length, white balance, luminance, contrast and brightness). Acquisition of the shooting information enables making the settings be substantially the same in every shooting, and thus, a similar image can always be acquired for a same shooting subject (object).

Here, when it is determined that a shooting condition information acquisition instruction signal has been received, the operation proceeds to the processing in next step S310. Also, if it is determined that no shooting condition information acquisition instruction signal has been received, the operation proceeds to the processing in step S311.

In step S310, the control section 31 transmits corresponding shooting condition information in view of the request from the first terminal 10.

In this case, the shooting condition information transmitted from the file server 30 to the first terminal 10 is a data file in which, e.g., the predetermined shooting conditions according to, e.g., the symptom, the disease state and/or the diseased part of the subject patient and various settings for the image pickup section 11 of the first terminal 10 are collected. Therefore, the file server 30 transmits proper shooting condition information in consideration of, e.g., specification information of respective image pickup sections 11 of the first terminal 10 that has accessed the file server 30 and the second terminal 20 related to the first terminal 10, and the subject patient information. Here, the specification information of the second terminal 20 is information acquired in the later-described processing in step S332 (image pickup specification information). In other words, the file server 30 also accumulates and holds, e.g., respective pieces of image pickup specification information of a plurality of information terminal apparatuses (10, 20) that access the file server 30. Subsequently, the operation proceeds to the processing in step S311.

In step S311, the control section 31 monitors an instruction signal from the first terminal 10, via the communication section 35 to determine whether or not a return instruction signal has been generated. Here, if it is determined that a return instruction signal has been generated, the operation returns to the above-described processing in step S301 and repeats the subsequent processing. Also, if it is determined that no return instruction signal has been generated, the operation returns to the above-described processing in step S304 and repeats the subsequent processing.

On the other hand, if it is determined in step S302 above that a terminal other than the first terminal 10 (hereinafter referred to as “second terminal 20”) has accessed the file server 30, the operation proceeds to the processing in step S331 and in step S331, the control section 31 performs predetermined login procedure processing (processing that is similar to the above-described processing in step S304 and step S312). In response to the processing, the second terminal 20 performs predetermined login procedure processing. Consequently, upon the login to the file server 30 being completed (if the login OK), the control section 31 performs processing for acquiring a medical reference image (watching-over image) transferred from the second terminal 20.

Although illustration is omitted also here, if the second terminal 20 cannot login to the file server 30, the file server 30 cancels the subsequent processing and returns to the above-described processing in step S301 and enters a standby state, which is similar to the above-described processing in each of step S304 and step S312.

In step S332, the control section 31 acquires the specification information of the image pickup section 11 from the second terminal 20. In this case, the specification information of the image pickup section 11 (hereinafter referred to as image pickup specification information) of the second terminal 20 is, for example, information indicating performance of the image pickup function such as setting values such as a shutter speed and an aperture value that can be set in the image pickup section 11, optical system focal length information (zoom range), an allowed shooting distance range (minimum shooting distance). As a result of the file server 30 holding the image pickup specification information, the file server 30 can set proper shooting conditions for acquiring a patient image using the second terminal 20.

Next, in step S333, the control section 31 associates the medical reference image (watching-over image) transferred from the second terminal 20 in the above-described processing in step S331 with a corresponding patient information file in the patient information database 34, based on medical meta data accompanying the image data, and records the medical reference image in the image database 32.

Next, in step S334, the control section 31 performs processing for comparing the medical reference image recorded this time (image data acquired in step S331 and recorded in the image database 32 in step S333) with a medical reference image (patient image data) of the same patient, the medical reference image being recorded last time. This image comparison processing is performed with reference to, for example, image change pattern model information created in steps S324 and S325 below.

Subsequently, in step S335, the control section 31 performs processing for determining whether or not an abnormal sign is observed as a result of a result of the image comparison processing in processing in step S334 above. Here, if it is determined that an abnormal sign is observed, the operation proceeds to the processing in next step S336. Also, if no abnormal sign is observed, the operation returns to the processing in step S301 and repeats the subsequent processing.

In step S336, the control section 31 access the first terminal 10 via the communication section 35 and transmits information on the abnormal sign determined this time to the first terminal 10. Subsequently, the operation returns to the processing in step S301 and repeats the subsequent processing.

On the other hand, in the processing in step S301 above, if it is determined that a terminal apparatus other than the external information terminal apparatuses (10, 20) has accessed the file server 30, the operation proceeds to the processing in step S321, and in step S321, the control section 31 determines whether or not the access is access from an internal terminal apparatus (not illustrated; for example, what is called an in-house terminal such as a PC installed at a consultation room of a hospital). Here, if the access is access from the internal terminal apparatus, the operation proceeds to the processing in next step S322. Also, if the access is not access from an internal terminal apparatus, the operation proceeds to the processing in step S323.

In step S322, the control section 31 performs predetermined login procedure processing (processing similar to the processing in each of step S304, step S312 and step S331 above). In response to the processing, the internal terminal apparatus (not illustrated) performs predetermined login procedure processing. Consequently, upon the login to the file server 30 being completed (if the login OK), the control section 31 transfers predetermined information to the internal terminal apparatus in response to a request from the internal terminal apparatus. Upon receipt of the predetermined information, the internal terminal apparatus displays the predetermined information on a display section (not illustrated). Also, in response to an input of information from the internal terminal apparatus, the control section 31 performs information recording processing (for example, an input of an electronic medical record update). Subsequently, the operation proceeds to the processing in step S323.

Although illustration is omitted also here, if the internal terminal apparatus cannot login to the file server 30, the file server 30 cancels the subsequent processing and returns to the processing in step S301 above and enters a standby state, which is similar to the processing in each of step S304, step S312 and step S331 above.

In step S323, in response to an instruction from the internal terminal apparatus, the control section 31 performs processing for registering and setting processing for an information terminal apparatus (10, 20). Subsequently, the operation returns to the processing in step S301 and repeats the subsequent processing.

If the internal terminal apparatus has not accessed from the file server 30 in the processing in step S321 above and the operation proceeds to the processing in step S324, in step S324 and next step S325, the control section 31 performs other processing steps.

For example, examples of the processing as the processing in step S324 and step S325 are as follows.

First, in step S324, when the control section 31 performs comparison between the latest image data and the last image data for the patient image data (medical reference images and watching-over images) of the subject patient recorded in the image database 32, the control section 31 performs deep learning based on a degree of change of a subject included in the images and a result of determination performed by the doctor based on the images (referring to electronic medical record information of the subject patient).

In next step S325, the control section 31 creates image change pattern model information based on a result of the deep learning performed in the processing in step S324 above. Then, the image change pattern model information is recorded and accumulated in, e.g., the image database 32. Subsequently, the operation returns to the processing in step S301 and repeats the subsequent processing.

The image change pattern model information thus created is used in, e.g., the image comparison processing in, e.g., the processing in step S334 above.

As described above, according to the above embodiment, the file server 30 including the patient information database 34, the file server 30 being connected to the network 40, and the information terminal apparatuses (the first terminal 10 and the second terminal 20) configured to communicate with the file server 30 via the network 40 configure the medical support system 1 for supporting medical service via patient information sharing.

In the medical support system 1, upon a doctor transmitting a predetermined order to the file server 30 using an information terminal apparatus (first terminal 10), the order information creating section 31b in the file server 30 creates order information based on the doctor's order. Based on the order information, the shooting condition information creating section 31c in the file server 30 creates shooting condition information according to the order information. The shooting condition information thus created is transmitted to an information terminal apparatus (second terminal 20).

Upon receipt of the shooting condition information, a person that is not a health professional, such as a patient's family member, performs image shooting using the information terminal apparatus (second terminal 20) to acquire image data of a subject patient. Consequently, the acquired image data can be approved as a medical reference image.

As described above, if order information including medical practice instructions provided by a doctor includes an image shooting instruction, the medical support system 1 according to the present embodiment creates proper shooting condition information for the image shooting instruction and transmits the proper shooting condition information to the information terminal apparatus.

Consequently, the doctor can refer to a patient image (reference image) having a desired form just by providing order information including normal medical practice instructions. Also, an image shooter who shoots a predetermined patient image based on the order information from the doctor can easily and reliably acquire a patient image according to the order information from the doctor even if the image shooter has neither high shooting skills nor predetermined shooting expertise, etc.

The medical support system 1 according to the present embodiment can handle not only image information that is patient information acquired by a health professional but also image information that is patient information acquired by a general person that is not a health professional (non-health professional), as a medical image or a medical reference image, enabling enhancement in ease of medical practice.

In this case, it is possible to create an image file having a form in which basic information (order information and shooting conditions for shooting) is linked with an image at the time of shooting, enabling prevention of misdiagnosis, enhancement in reliability of the information and facilitation of management of valuable image information. Also, as necessary, the medical support system 1 according to the present embodiment can be made to a system configured so that medical basic information is provided to an image file.

Also, in an information terminal apparatus, patient basic attribute information is acquired in advance as, e.g., “supplementary information for management”, “supplementary information for medical examination”, “medical examination management information”, and thus, at the time of shooting using the information terminal apparatus, these pieces of information can be used as supplementary information for an acquired image.

Consequently, at the time of creation of an image file, these pieces of information can be provided as supplements to the image file, and thus, even if the image data is brought alone to the outside from the system or a particular folder, the image can be determined in terms of when and where the image was shot and whose test result the image is, with reference to the supplementary tag information.

For example, an order number assigned to order information is linked with, e.g., a test ID, enabling association with a series of test studies and a particular disorder/disease test.

A simple photo alone indicates neither a patient name nor a purpose of the photo, but these pieces of information supplementary to an image file ensures usefulness as an image for medical examination.

For example, for image data used by, e.g., a patient as a reference for a chief complaint or image data managed by a non-health professional for himself/herself, it is not necessary to acquire order information. However, if these image data are contemplated for being included in electronic medical records in a medical facility the patient visits regularly or may be useful as reference data for medical examination, it is effective to cause formats and data of supplementary information such as “supplementary information for management”, “supplementary information for medical examination” and “medical examination management information” to be received in an information terminal apparatus in advance.

In other words, in the present embodiment, the statement “order information” is not limited to an order in a strict sense and includes information that can be called “supplementary information for management”, “supplementary information for medical examination” or “medical examination management information”. Then, each information terminal apparatus described in the present embodiment is configured to receive information necessary for enhancement in efficiency and reliability of image data when the image data is submitted to a medical facility, from the medical facility (system) and create image data based on the information.

Medical support systems conventionally used and disclosed are intended for a health professional to collectively manage patient information, etc., at a medical base such as a hospital. Furthermore, no consideration has been made on such system being used by a person other than a main health professional such as an attending doctor. Also, in conventional medical support systems, no consideration has been made on handling medical images each being patient information in any way. However, the technique according to the present invention enables medical information, which has been acquired in a dedicated environment or facility according to an instruction provided by a doctor to a health professional and/or a patient, to be released from the conventional constraints and be acquired in a more open environment. Consequently, it is possible to catch a rare but important symptom that appears in a particular environment only.

In general, it is premised that only image information acquired based on prescribed settings and format by a person having a predetermined healthcare-related qualification in response to a doctor's order (a request, an instruction or the like) is approved as a medical image formally recognized as patient information. However, the present invention can eliminate this premise. In other words, the need for a patient to spend time and the need for providing a particular environment can be eliminated. A person who is not a health professional (general user), a health professional other than an attending doctor or a health professional other than a hospital official can acquire patient information as important information, e.g., in a patient's living environment or at a timing of the patient visiting a certain facility.

Therefore, even an image of a patient acquired by a person having no predetermined healthcare-related qualification (for example, a care giver such as a patient's family member or close relative who is a non-health professional), using a privately-owned image pickup apparatus, information terminal apparatus or the like can be handled as a formal medical image, and the person can acquire information in an optimum condition in consideration of the attending doctor's need, without having expertise. Also, it is possible to acquire information by, e.g., shooting in aware of the doctor's request. A characteristic of a shooting device according to this invention lies in that it is possible to acquire an image that can be used as a reference image for medical practice, for example, an image in which a patient-specific symptom is “shot by coincidence” even from the images acquired as usual commemorative photographs in everyday life of the patient or the shooter (patient's family member, etc.). Furthermore, as another Characteristic of a shooting device according to this invention lies in that, in a case where the patient's family member performs shooting based on the order information acquired from the doctor in everyday life spent with the patient, when the ordered conditions cannot be satisfied, the shooting device is capable of “providing a function for giving an advice such as guide information, warning display, etc.”, so as to enable the patient's family member to perform shooting that satisfies the ordered conditions. A shooting device according to the present invention enables acquisition of a medicate image in an open environment in such a manner that the acquisition is not necessarily constrained by an environment or a service situation.

In recent years, information terminal apparatuses each having an image pickup function that enables easy acquisition of a high-resolution image as well as a communication function, the information terminal apparatuses being compact and portable, more specifically, for example, smartphones, table PCs or the like, have been popularized. An environment in which by use of these information terminal apparatus, anyone can acquire a high-resolution image at any time and the acquired image can easily be shared with another person having an information terminal apparatus of a similar type has been provided. Therefore, the present invention is an invention that actively utilizes such infrastructure to ease constraints relating to medical service.

According to the present invention, an image acquired by not only a health professional but also a person that is not a health professional (a care giver, such as a patient's family member or close relative, that is, a non-health professional) using a popularized information terminal apparatus (for example, a communication function-equipped image pickup apparatus (camera) or an image pickup function-equipped communication apparatus (e.g., a smartphone or a tablet PC)) can be handled as a medical image or a medical reference image that is patient information, by reflecting doctor's requests in the image.

The present invention enables provision of a medical support system that can handle not only image information that is patient information, the image information being acquired in a hospital by a health professional in the hospital or a related health professional in response to an order from an attending doctor or the like, but also even image information that is patient information, the image information being acquired by a general person that is not a health professional (non-health professional) or a person equivalent to such general person, as a medical image or a medical reference image, and thus enhance ease of medical practice, an information terminal apparatus employed in the medical support system, a patient image data acquisition method in the medical support system and an image file format that is effective in such environment.

Although the above embodiment has been described taking, e.g., general portable image pickup apparatuses (camera), smartphones and tablet PCs each having an image pickup function and a communication function, as example forms of the information terminal apparatuses (10, 20), a form of information terminal apparatus that can be employed in the present invention is not limited to these forms.

For example, in recent years, a camera system (network camera system including a camera, such as what is called a watching-over camera or a web camera) including a camera installed at a predetermined site (for example, a subject patient's home or a patients room in a hospital or the like), the camera system being configured so that the camera can be remotely operated via a network to acquire shot images at predetermined time intervals, and the acquired shot image can be transferred to another device via the network in order to consistently watch over a status of, e.g., a subject patient subject to image shooting, has been put into practical use and becoming popularized. This type of camera system can also be employed as an information terminal apparatus according to the present invention.

The processing sequences described in the above embodiment can be changed in proceeding as long as such change is contrary to the nature of the processing sequence. Therefore, in each of the above processing sequences, for example, the order of respective processing steps to be performed may be changed, a plurality of processing steps may be performed simultaneously or the order of respective processing steps may be different each time a processing sequence is executed. In other words, even though the operation flows in the claims, the description and the drawing are described using terms such as “first” and “next” for sake of convenience, the use of such terms does not mean that it is essential to perform the operation flows in the stated order. Also, it should be understood that the respective steps included in these operation flows may arbitrarily be omitted as long as such omission does not affect the spirit of the invention.

Also, from among the techniques described here, many of the control and functions described mainly with reference to the flowcharts can often be set by software programs, and the above control and functions can be provided by the software programs being read and executed by a computer. Each of the software programs is electronic data, an entirety or a part of the electronic data being stored or recorded in, e.g., a storage medium or a storage section, more specifically, a storage medium, for example, a portable medium such as a flexible disk, a CD-ROM or a non-volatile memory, or a hard disk or a volatile memory, in advance in a product manufacturing process, as a computer program product. Also, aside from the above, the software programs can be distributed or provided at product shipment or via a portable medium or a communication channel. Even after product shipment, a user can make these software programs operate by downloading the software programs via, e.g., a communication network or the Internet and or installing the software programs in a computer from a storage medium, enabling easy provision of an image pickup apparatus according to the present embodiment.

Here, in the embodiment, each of parts stated as “section” or “unit” may be configured by a dedicated circuit or a combination of a plurality of general-purpose circuits, and as necessary, may be configured in a combination with a processor such as a microcomputer configured to operate according to a programmed software or a CPU or a sequencer such as an FPGA. Also, a design can be made so that a part or an entirety of control of the section is undertaken by an external apparatus, in this case, via a wired or wireless communication circuit. For communication, e.g., Bluetooth, WiFi or a telephone line may be employed or, e.g., USB may be employed. A dedicated circuit, a general-purpose circuit and/or a control section may be integrated to configure an ASIC. A moving section or the like includes any of various actuators and as necessary, a joining mechanism for movement, and each actuator is activated by a drive circuit. The drive circuit is controlled by, e.g., a microcomputer or an ASIC according to a particular program. Such control may be subjected to detailed correction, adjustment or the like based on information outputted by various sensors and/or peripheral circuits of the sensors.

It should be understood that the present invention is not limited to the above-described embodiment and that various modifications and applications are possible without departing from the spirit of the invention. Furthermore, the above embodiment includes various phases of the invention, and various aspects of the invention can be extracted by arbitrary combination of a plurality of components disclosed. For example, even if several components are deleted from all the components indicated in the above-described embodiment, the configuration with the components deleted can be extracted as an aspect of the invention as long as such configuration can solve the problem to be solved by the invention and provide effects of the invention. Furthermore, components in different embodiments may arbitrarily be combined. This invention is not limited by any particular embodiment of the invention except that the invention is limited by the accompanying claims.

What is claimed is:

1. A medical support system for supporting medical service via patient information sharing, the system comprising:
a file server including a patient information database, the file server being connected to a network; and
an information terminal apparatus configured to communicate with the file server via the network and transmit inputted order information to the file server,
wherein the file server creates corresponding shooting condition information based on the order information transmitted from the information terminal apparatus and transmits the shooting condition information and the order information to the information terminal apparatus.

2. The medical support system according to claim 1, wherein the information terminal apparatus performs image shooting based on the shooting condition information transmitted from the file server, and records acquired image data in association with the order information.

3. The medical support system according to claim 2, wherein the information terminal apparatus transfers the image data acquired by the image shooting to the file server.

4. The medical support system according to claim 1, wherein
the file server includes
an order information creating section configured to create order information upon receipt of an input of an instruction from the information terminal apparatus, and
a shooting condition information creating section configured to, based on the order information created by the order information creating section, create shooting condition information according to the order information, and
based on the order information from the order information creating section, the file server refers to the patient information database for patient information of a patient subject to the order information, to search the patient information of the subject patient for past order information, and if past order information similar to the relevant order information is found, reads shooting condition information relating to image shooting included in the past order information, and transmits the shooting condition information to the shooting condition information creating section, and upon receipt of the shooting condition information, the shooting condition information creating section creates new shooting condition information for the relevant order information.

5. The medical support system according to claim 1, wherein
the file server further includes an image database, and
the image database accumulates and stores a plurality of image data acquired by the information terminal apparatus.

6. The medical support system according to claim 5, wherein each of the plurality of image data accumulated in the image database is associated with a corresponding piece of patient information from among a plurality of pieces of patient information accumulated in the patient information database.

7. The medical support system according to claim 5, wherein
the image database further accumulates and stores a plurality of sample images shot based on shooting condition information determined in advance for respective predetermined shooting subjects, and
the shooting condition information creating section creates shooting condition information for the order information created by the order information creating section, with reference to the plurality of sample images in the image database.

8. An information terminal apparatus comprising:
an image pickup section configured to acquire image data;
a communication section configured to communicate with a file server via a network and transmit inputted order information to the file server; and
an image pickup control section configured to receive shooting condition information created based on the order information from the file server, performs image shooting according to the shooting condition information and associates the order information with the acquired image data.

9. The information terminal apparatus according to claim 8, wherein the image pickup control section transfers the acquired image data to the file server.

10. The information terminal apparatus according to claim 8, further comprising a display section configured to perform display of an image based on the image data, and various types of setting information,
wherein during image shooting performed according to the shooting condition information, in a case where the shooting condition information fails to be met, the image pickup control section displays guide information or warning information of the failure on the display section.

11. The information terminal apparatus according to claim 10, wherein the case where the shooting condition information fails to be met is a case where shooting based on shooting condition information corresponding to the order information fails to be performed.

12. The information terminal apparatus according to claim 10, wherein the case where the shooting condition information fails to be met is a case where consistency with medical examination management information fails to be ensured.

13. The information terminal apparatus according to claim 10, wherein determination of whether or not the shooting condition information is met is performed by comparing numeral values obtained by quantifying results of shooting according to respective conditions, with a predetermined criterion, the respective conditions including:
a condition that a shooting subject person or a shooting subject region is different;
a condition that contemplated equipment or accessory is not used;
a condition that a designated composition is not achieved;
a condition that a shooting parameter fails to be set;
a condition that an amount of exposure is insufficient;
a condition that no proper focusing can be performed;
a condition that a camera shake occurs;
a condition that a sequence of shooting is not a prescribed sequence; and
a condition that a number of shot images is larger or smaller than a designated number of images.

14. The information terminal apparatus according to claim 10, wherein determination of whether or not the shooting condition information is met is performed based on determination criteria including a result of patient identification based on voice information or other intention indication and whether or not shooter information is included.

15. The information terminal apparatus according to claim 10, wherein the determination of whether or not the shooting condition information is met is performed before or after shooting based on the order information is performed.

16. A method for acquiring patient image data included in patient information in a medical support system for supporting medical service via patient information sharing, the method comprising:
creating order information in response to a doctor's order;
creating shooting condition information according to the order information;
transmitting the shooting condition information to an information terminal apparatus; and
causing the information terminal apparatus to acquire corresponding image data in response to the shooting condition information.
